(19) United States
(12) Patent Application Publication
(10) Pub. No.: US 2024/0138773 A1
(43) Pub. Date: May 2, 2024

(54) SEQUENTIALLY-REDUCED ARTIFICIAL INTELLIGENCE BASED SYSTEMS AND METHODS FOR CARDIOVASCULAR TRANSFER FUNCTIONS

(71) Applicant: University of Southern California, Los Angeles, CA (US)

(72) Inventors: Niema Mohammed PAHLEVAN, Los Angeles, CA (US); Rashid Alavi DEHKORDI, Los Angeles, CA (US); Faisal AMLANI, Los Angeles, CA (US); Hossein GORJI, Zürich (CH); Soha NIROUMANDIJAHROMI, Los Angeles, CA (US); Heng WEI, Los Angeles, CA (US)

(21) Appl. No.: 18/499,037

(22) Filed: Oct. 31, 2023

Related U.S. Application Data
(60) Provisional application No. 63/381,671, filed on Oct. 31, 2022.

(51) Int. Cl. A61B 5/00; A61B 5/021; A61B 5/0215; A61B 5/1455; G16H 10/60; G16H 50/20; G16H 50/30; G16H 70/60

(52) U.S. Cl. CPC A61B 5/7267; A61B 5/0031; A61B 5/02108; A61B 5/0215; A61B 5/14551; A61B 5/6852; A61B 5/7257; G16H 10/60; G16H 50/20; G16H 50/30; G16H 70/60

(57) ABSTRACT

Systems, methods, devices, and machine readable media storing instructions (programming) for an instantaneous or nearly instantaneous (e.g., within 0.1 seconds, within 0.001 seconds, etc.), non-invasive, and easy-to-use transfer from a radial and/or brachial waveform to a carotid waveform or its reduced-order parameters are described. Some embodiments relate to systems, methods, devices, and programming for determining cardiovascular (clinical) indices and biomarkers from two or more of the radial and/or brachial and/or carotid waveforms (or their corresponding reduced-order representations).

### BACKGROUND

### 1. Field

The present disclosure relates generally to sequentially-reduced artificial intelligence based systems and methods for cardiovascular transfer functions.

### 2. Description of the Related Art

The extraction of waveforms and cardiovascular indices from various clinical measurements are often formulated as inverse/minimization (parameter estimation) problems that are traditionally very computationally expensive to solve as they require repeated calculation of the forward Navier-Stokes models in approximated physical configurations. The ultimate goal is to enable instantaneous/real-time extraction and subsequent analysis for use by clinicians. General-purpose function approximators that have been established by machine learning (ML) offer new avenues for such endeavors. Their speed, accuracy, robustness, and universality make them appropriate building blocks for remote health monitoring and early diagnosis. The possibility of employing ML algorithms in assisting the diagnosis of cardiovascular diseases has led to an interest in reliable yet efficient classification models of cardiovascular waveforms.

### SUMMARY

The following is a non-exhaustive listing of some aspects of the present techniques. These and other aspects are described in the following disclosure.

Some aspects relate to systems, methods, devices, and machine readable media storing instructions (programming) for an instantaneous or nearly instantaneous (e.g., within 0.1 seconds, within 0.001 seconds, etc.), non-invasive, and easy-to-use transfer from a radial and/or brachial waveform to a carotid waveform or its reduced-order parameters. Some embodiments relate to systems, methods, devices, and programming for determining cardiovascular (clinical) indices and biomarkers from two or more of the radial and/or brachial and/or carotid waveforms (or their corresponding reduced-order representations).

For example, some aspects include a method comprising receiving, by inputs of a trained sequentially-reduced feedforward neural network (FNN) model, patient data having one or more cardiovascular waveforms (i.e., radial and/or brachial pressure or vessel wall displacement waveforms in any order) broken down to 50-5000 discrete datapoints. The method comprises determining, via the trained sequentially-reduced FNN model, from one or more of the one or more cardiovascular waveforms, a pressure waveform corresponding to the carotid artery (or vessel wall displacement waveform of the carotid artery). The method comprises, responsive to determining the carotid waveform, providing, to a user, underlying pathology information revealed by carotid artery information indicated by the pressure waveform corresponding to the carotid artery.

In some embodiments, the one or two input waveforms (e.g., a radial waveform, or a brachial waveform) to the sequentially-reduced FNN model are inputted by or in different orders. The different orders comprise: (i) only radial, (ii) only brachial, (iii) first radial, then brachial, and (iv) first brachial, then radial.

In some embodiments, the outputs of the sequentially-reduced FNN model are the reduced-order parameters corresponding to at least one of: the carotid pressure waveform or vessel wall displacement of the carotid artery, the reduced-order parameters including intrinsic frequencies, augmentation indices, wave intensity parameters, form factor, pulse pressure amplification, travel time of the reflected wave, or Fourier transform representation components.

In some embodiments, the intrinsic frequencies comprise a double frequency version or a multiple harmonic intrinsic frequency version.

In some embodiments, wave intensity parameters comprise at least one of a first forward peak/time, a first backward peak/time, or a second forward peak/time.

In some embodiments, the inputs are the reduced-order representation of the waveforms using any basis function expansion, Fourier transform representation, or the intrinsic frequency representation of the waveform(s).

In some embodiments, the basis function expansion comprises eigenfunctions.

In some embodiments, the Fourier transform representation is truncated by one or more different frequencies.

In some embodiments, any short-time Fourier transform, windowed Fourier transform, or wavelet transform is used to provide as input such reduced-order representations and expansions based on subdivided segments of a waveform.

In some embodiments, the method comprises steps for training the trained sequentially-reduced FNN model.

In some embodiments, Fourier transform representation components comprise the amplitude and/or phase of sinusoidal components with different frequencies.

In some embodiments, the method comprises, during steps for model training, utilization of Fourier-based custom loss functions designed to incorporate a reconstructed weighted waveform, a waveform's second derivative weighted or, the reconstructed waveform's second derivative weighted reduced-order parameters from input and output waveforms, or combinations thereof.

In some embodiments, model architecture comprises one or more FNNs, artificial neural networks (ANNs), recurrent neural networks (RNNs), temporal convolutional neural networks (TCNNs), and/or Random Forest Regressors (RFRs), and/or other architectures.

In some embodiments, reduced-order parameters utilized in Fourier-based custom loss functions comprise any number of reduced-order parameters (e.g., any number components from the Fourier transform representation components from the first 10 to the first 25 components, or any other desired number).

In some embodiments, input and output waveforms utilized in the Fourier-based custom loss functions for generating reduced-order parameters comprise patient data having two or more cardiovascular waveforms including: radial and/or brachial and/or carotid pressure and/or vessel wall displacement waveforms, in any order.

In some embodiments, the method comprises implementation of short-time Fourier transform-based or windowed Fourier-transform-based representation methods during generating of reduced-order parameters utilized in Fourier-based custom loss functions.

In some embodiments, the method comprises application of short-time Fourier transform-based or windowed Fourier-transform-based methods on any segment of a waveform, including diastolic, systolic, or any other desired time-interval or subdivided segment within the cardiac cycle.

As another example, some aspects include a method comprising receiving, as inputs of a trained artificial intelligence (AI) model, patient data having one, two, or more cardiovascular waveforms broken down to 50-5000 discrete datapoints. The method comprises determining, utilizing the trained AI model, from two or more waveforms, the patient's cardiovascular indices such as cardiac output, carotid-femoral pulse wave velocity, LV stroke volume, LV filling pressure (e.g., LV end diastolic pressure), LV contractility (e.g., LV ejection fraction, fractional shortening, LV end systolic elastance), aortic characteristic impedance, arterial compliance, LV compliance, and LV-aortic coupling indices as well as cardiovascular-affecting disease indices such as HOMA index (for diabetes). The method comprises, as a result, determining the underlying pathology information revealed by such parameters to a user.

In some embodiments, the trained AI model comprises a trained sequentially-reduced feedforward neural network (FNN) model.

In some embodiments, the patient data having two or more cardiovascular waveforms comprises: radial pressure waveforms, brachial pressure waveforms, carotid pressure waveforms, radial vessel wall displacement waveforms, brachial vessel displacement waveforms, and/or carotid vessel displacement waveforms.

In some embodiments, the two or more cardiovascular waveforms comprise patient data having two or more cardiovascular waveforms, such as radial and/or brachial and/or carotid pressure and/or vessel wall displacement waveforms in any order in any order.

In some embodiments, the two or more input waveforms to the sequentially-reduced FNN model are inputted in different orders. The different orders comprising at least one of: (i) first radial, second brachial, (ii) first brachial, second radial, (iii) first radial, second carotid, (iv) first carotid, second radial, (v) first brachial, second carotid, (vi) first carotid, second brachial, (vii) first radial, second brachial, third carotid, (viii) first radial, second carotid, third brachial, (ix) first brachial, second radial, third carotid, (x) first brachial, second carotid, third radial, (xi) first carotid, second radial, third brachial, or (xii) first carotid, second brachial, third radial.

In some embodiments, one or more waveforms are from a pulse oximeter measurement or femoral waveform.

In some embodiments, the trained AI model comprises: a recurrent neural network (RNN), a temporal convolutional neural network (TCNN), or a Random Forest Regressor (RFR).

In some embodiments, the method comprises providing the trained AI model to a client device having a diagnosis module configured to execute the trained AI model to perform the determination of a specific cardiovascular disease via the client device.

The client device may be a smartphone, microwave-based device, or a wearable device. In some embodiments, the client device includes at least one sensor configured to measure an arterial blood pressure of a patient, a pulse rate of the patient, a pulse-ox of the patient, and/or an arterial wall displacement of the patient. In some embodiments, the client device comprises an electrocardiogram (ECG) device configured to capture an ECG of the patient. In some embodiments, the client device is an implantable wireless system. In some embodiments, the client device is an invasive arterial line.

Some aspects include a tangible, non-transitory, machine-readable medium storing instructions that when executed by a data processing apparatus cause the data processing apparatus to perform operations of the above-mentioned process(es).

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 is a logical-architecture block diagram that illustrates a sequentially-reduced artificial intelligence (AI) model-based system including a transfer engine and other components for cardiovascular transfer functions, in accordance with various embodiments.

FIG. 2 illustrates an example of a sequentially reduced feedforward neural network, in accordance with various embodiments.

FIG. 3 shows an example block diagram of operations for applying cardiovascular transfer functions, in accordance with various embodiments.

FIG. 4 illustrates a flowchart describing determination of intrinsic frequency (IF) methodology parameters based on an input carotid waveform (as one example input waveform), in accordance with various embodiments.

FIG. 5 illustrates determining a second (or target) waveform determined based on a first (input) waveform, in accordance with various embodiments.

FIG. 6 provides a graph illustrating a sensitivity of the accuracy of an embodiment of the AI model described herein to training data set size, in accordance with various embodiments.

FIG. 7 is a schematic illustration of a trained sequentially-reduced AI model for predicting (reduced-order) IF parameters from a single input carotid pressure waveform, and the dicrotic notch time, as two representative examples of an input waveform and reduced-order parameters, respectively, in accordance with various embodiments.

FIG. 8 illustrates example evaluation plots for a representative embodiment of the AI model (e.g., the AI model shown in FIG. 7) described herein, in accordance with various embodiments.

FIG. 9 is a diagram that illustrates an exemplary computing system, in accordance with various embodiments.

FIG. 10 is a flow chart that illustrates a sequentially-reduced artificial intelligence based process performed by the transfer engine and other components shown in FIG. 1 for cardiovascular transfer functions, in accordance with various embodiments.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the field of waveform and cardiovascular indicia extraction from various clinical measurements. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

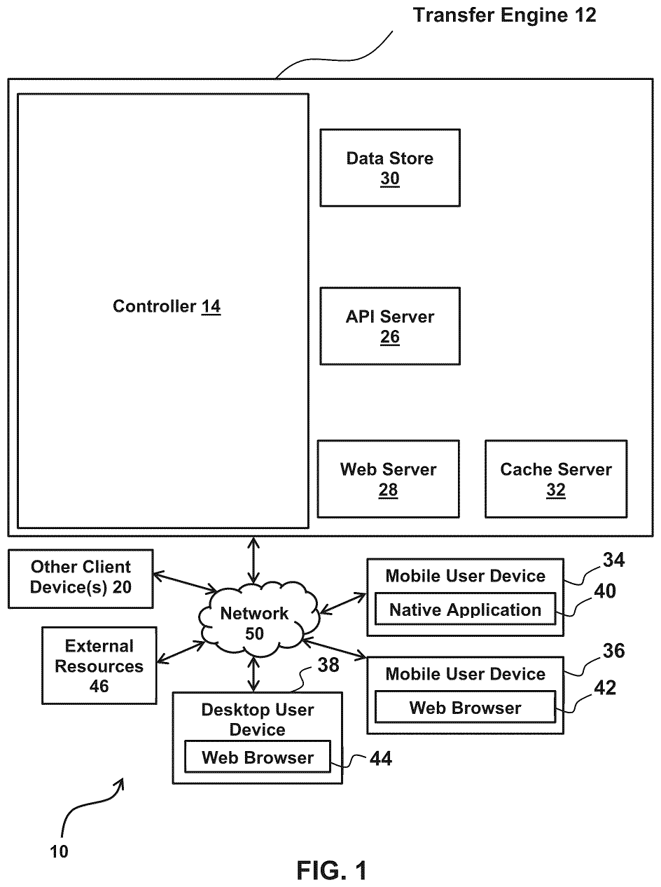

FIG. 1 illustrates a system 10 comprising a transfer engine 12 and other components configured to provide sequentially-reduced artificial intelligence (AI) based tools for cardiovascular transfer functions. A transfer function can be used to transfer or convert a first dataset into a second dataset (or otherwise determine the second dataset based on the first dataset). An AI model may be used for such a transfer or conversion. As described herein, a dataset may comprise a waveform (several datapoints collected over time that form some (usually non-linear) shape if plotted graphically). Transfer or conversion of one dataset or waveform to another (or otherwise determining a second data set or waveform based on a first dataset or waveform), or to a set of reduced-order parameters associated with a waveform (e.g., discretized pressure waveform, the components of Fourier or windowed-Fourier transform representation of the waveform) may be beneficial to facilitate better visualization and/or analysis of data, among other advantages.

When transferring or otherwise converting a waveform comprising 50 to 5000 or more discrete datapoints, for example, into another waveform and/or reduced-order parameters of another anatomical site's waveform using an AI model, there is often a large difference between the sizes of the inputs and outputs from an AI model. For example, an input vector to a model may be of high dimension (e.g., 100 or more dimensions, 1,000 or more dimensions, 100,000 or more dimensions, etc.), while the output vector is of low dimension (e.g., less than 100 dimensions, less than 10 dimensions, etc.). System 10 is configured to generate, train, and/or otherwise utilize an AI model comprising one or more neural networks that benefit from a sequentially converging structure, as described below. For example, a sequentially converging structure may have a number of neurons in each layer systematically decreases from the input layer to the output layer (e.g., layer 1 has N dimensions, layer 2 has N−1 dimensions, and so on). Utilizing such a model can improve the globalization and robustness of the model's function as well as facilitating easier training of the AI model, and provide an instantaneous or near instantaneous (e.g., within 0.1 seconds, within 0.001 seconds, etc.), non-invasive, and easy-to-use transfer from one waveform (e.g., a radial and/or brachial waveform) to another (e.g., a carotid waveform), or its reduced-order parameters to facilitate better visualization and/or analysis of data. Such a mode may also be used for determining cardiovascular (clinical) indices and biomarkers from two or more of the radial and/or brachial and/or carotid waveforms (or their corresponding reduced-order representations).

Embodiments of system 10 may have a number of practical applications, and may provide a number of real-world technical solutions to existing technical problems. Some example non-limiting applications include:
non-invasive and instantaneous determination of the carotid waveform, thereby extracting its clinically significant outputs for use in appropriate therapy and preventive medicine as well as for providing the underlying pathology information contained in the carotid artery;
semi-invasive and beat-to-beat monitoring of heart failure (HF) development in hospitals or clinical environments from clinically significant outputs;
non-invasive and instantaneous detection of cardiovascular diseases from clinically significant outputs;
semi-invasive and beat-to-beat monitoring and management of HF status in hospitals or clinical environments;
discharge management of treated patients from hospitals;
monitoring the compensated state of patients after discharge; and
evaluating and predicting effects of different preventive/curative drugs related to cardiovascular diseases.
   Other applications are also possible.

These and other benefits are described in greater detail below, after introducing the components of system 10 and describing their operation. It should be noted, however, that not all embodiments necessarily provide all of the benefits outlined herein, and some embodiments may provide all or a subset of these benefits or different benefits, as various engineering and cost tradeoffs are envisioned, which is not to imply that other descriptions are limiting.

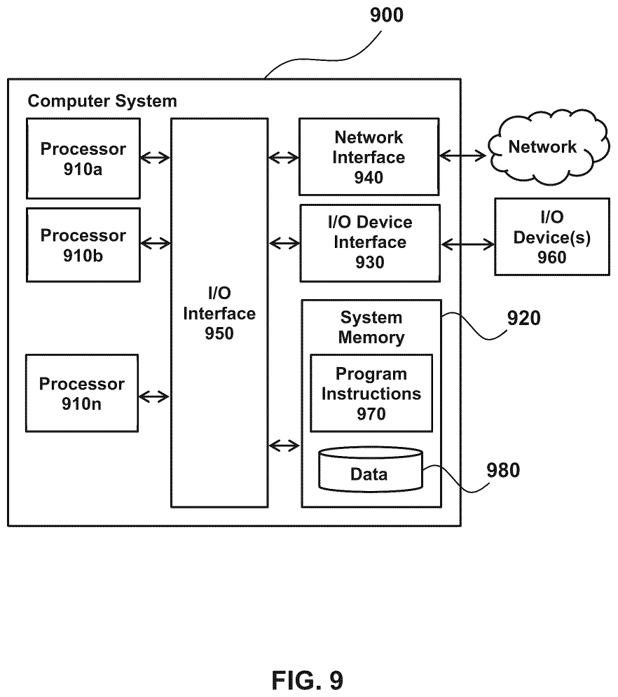

In some embodiments, transfer engine 12 is executed by one or more of the computers described below with reference to FIG. 9 and includes an application program interface (API) server 26, a web server 28, a data store 30, and a cache server 32. These components, in some embodiments, communicate with one another in order to provide the functionality of transfer engine 12 described herein. As described in greater detail below, in some embodiments, data store 30 may store data including patient medical information (e.g., in the form of an electronic medical record); patient biographical information; clinical measurements; test results; sensor data; cardiovascular waveforms, indices, and/or data indicative of such waveforms and/or indices; reduced-order parameters associated with one or more of the waveforms weights associated with different types of information; relational data; artificial intelligence models and/or instructions for training and/or executing such models; and/or other information.

Cache server 32 may expedite access to this data by storing likely relevant data in relatively high-speed memory, for example, in random-access memory or a solid-state drive. Web server 28 may serve webpages having graphical user interfaces that display login views, one or more views that facilitate transfer of one waveform to another, and/or to reduced-order parameters of another anatomical site's waveform, one or more views that facilitate obtaining information from a patient, a sensor, or other sources; one or more views that facilitate data analysis potentially including making a medical diagnosis for example (e.g., after a transfer is complete); or other displays. API server 26 may serve data to various applications that process data related to user logins, the transfer of one waveform to another, and/or to reduced-order parameters of another anatomical site's waveform, the information obtained from a patient, analyzed data, data from a sensor, or other sources, or other data. The operation of these components 26, 28, and 30 may be coordinated by a controller 14, which may bidirectionally communicate with each of these components or direct the components to communicate with one another. Communication may occur by transmitting data between separate computing devices (e.g., via transmission control protocol/internet protocol (TCP/IP) communication over a network), by transmitting data between separate applications or processes on one computing device; or by passing values to and from functions, modules, or objects within an application or process, e.g., by reference or by value.

Among other operations, in some embodiments, transfer engine 12 trains an artificial intelligence model using training data that describes prior waveform transfers, for example, and/or other training information. Transfer engine 12 receives new waveforms and corresponding requests for transferring the waveforms and instantaneously (or nearly instantaneously) determines, with the model, waveform transfers.

In some embodiments, interaction with users (e.g., medical practitioners such as doctors or other practitioners), patients, and/or other entities (e.g., a healthcare provider) may occur via a website or a native application viewed on a desktop computer, tablet, or a laptop of the user. In some embodiments, such interaction occurs via a mobile website viewed on a smart phone, tablet, or other mobile user device, or via a special-purpose native application executing on a smart phone, tablet, or other mobile user device.

To illustrate an example of the environment in which transfer engine 12 operates, the illustrated embodiment of FIG. 1 includes a number of components with which transfer engine 12 communicates: mobile user devices 34 and 36; a desk-top user device 38; (other) client devices 20; and external resources 46. Each of these devices typically communicates with transfer engine 12 via a network 50, such as the Internet or the Internet in combination with various other networks, like local area networks, cellular networks, Wi-Fi networks, or personal area networks (though it is possible to configure system 10 such that one or more of these components communicate via wires).

Mobile user devices 34 and 36 may be smart phones, tablets, smart watches, wearable devices, gaming devices, or other hand-held networked computing devices having a display, a user input device (e.g., buttons, keys, voice recognition, or a single or multi-touch touchscreen), memory (such as a tangible, machine-readable, non-transitory memory), a network interface, a portable energy source (e.g., a battery), and a processor (a term which, as used herein, includes one or more processors) coupled to each of these components. The memory of mobile user devices 34 and 36 may store instructions that when executed by the associated processor provide an operating system and various applications, including a web browser 42 or a native mobile application 40. The desktop user device 38 may also include a web browser 44. In addition, desktop user device 38 may include a monitor; a keyboard; a mouse; memory; a processor; and a tangible, non-transitory, machine-readable memory storing instructions that when executed by the processor provide an operating system and the web browser. Native application 40 and web browsers 42 and 44, in some embodiments, are operative to provide a graphical user interface associated with a user (e.g., a medical practitioner such as a doctor), a patient, and/or a medical services provider, for example, that communicates with transfer engine 12 and facilitates user, patient, and/or medical services provider interaction with data from transfer engine 12. Web browsers 42 and 44 may be configured to receive a website from transfer engine 12 having data related to instructions (for example, instructions expressed in JavaScript™) that when executed by the browser (which is executed by the processor) cause mobile user device 36 and/or desktop user device 38 to communicate with transfer engine 12 and facilitate user, patient, and/or medical services provider interaction with data from transfer engine 12. Native application 40 and web browsers 42 and 44, upon rendering a webpage and/or a graphical user interface from transfer engine 12, may generally be referred to as client applications of transfer engine 12, which in some embodiments may be referred to as a server. Other client devices 20 may include one or more sensors such as a pulse oximeter, a microwave-based device, a wearable device, an implantable wireless system, an invasive arterial line, and/or other devices, for example.

Embodiments, however, are not limited to client/server architectures, and transfer engine 12, as illustrated, may include a variety of components other than those functioning primarily as a server. Three user devices and one other client device are shown, but embodiments are expected to interface with substantially more, with more than 100 concurrent sessions and serving more than 1 million users distributed over a relatively large geographic area, such as a state, the entire United States, and/or multiple countries across the world.

External resources 46, in some embodiments, include sources of information such as databases, websites, etc.; external entities participating with the system 10 (e.g., systems or networks associated with healthcare or medical service providers), one or more servers outside of the system 10, a network (e.g., the internet), electronic storage, equipment related to Wi-Fi™ technology, equipment related to Bluetooth® technology, data entry devices, sensors, network accessible medical equipment, or other resources. In some embodiments, external resources 46 include one or more other client devices 20, and vice versa. In some implementations, some or all of the functionality attributed herein to external resources 46 may be provided by resources included in the system 10. External resources 46 may be configured to communicate with transfer engine 12, mobile user devices 34 and 36, desktop user device 38, other client devices 20, and/or other components of the system 10 via wired and/or wireless connections, via a network (e.g., a local area network and/or the internet), via cellular technology, via Wi-Fi technology, and/or via other resources.

Thus, transfer engine 12, in some embodiments, operates in the illustrated environment by communicating with a number of different devices and transmitting instructions to various devices to communicate with one another. The number of illustrated external resources 46, desktop user devices 38, mobile user devices 36 and 34, and other client devices 20 is selected for explanatory purposes only, and embodiments are not limited to the specific number of any such devices illustrated by FIG. 1, which is not to imply that other descriptions are limiting.

Transfer engine 12 of some embodiments includes a number of components introduced above that provide a sequentially-reduced artificial intelligence based tool for cardiovascular transfer functions. For example, the illustrated API server 26 may be configured to communicate data about users, an input waveform, a transferred waveform, and/or other information via a protocol, such as a representational-state-transfer (REST)-based API protocol over hypertext transfer protocol (HTTP) or other protocols. Examples of operations that may be facilitated by the API server 26 include requests to display, link, modify, add, or retrieve portions or all of such waveform data, or other information. API requests may identify which data is to be displayed, linked, modified, added, or retrieved by specifying criteria for identifying records, such as queries for retrieving or processing information about a particular input or output waveform (or additional user information associated with a waveform), for example. In some embodiments, the API server 26 communicates with the native application 40 of the mobile user device 34 or other components of system 10.

The illustrated web server 28 may be configured to display, link, modify, add, or retrieve portions or all of patient medical information (e.g., in the form of an electronic medical record); patient biographical information; clinical measurements; test results; sensor data; cardiovascular waveforms, indices, and/or data indicative of such waveforms and/or indices; reduced-order parameters associated with one or more of the waveforms weights associated with different types of information; relational data; artificial intelligence models and/or instructions for training and/or executing such models; and/or other information encoded in a webpage (e.g. a collection of resources to be rendered by the browser and associated plug-ins, including execution of scripts, such as JavaScript™, invoked by the webpage). In some embodiments, the graphical user interface presented by the webpage may include inputs by which the user may enter or select data, such as clickable or touchable display regions or display regions for text input. Such inputs may prompt the browser to request additional data from the web server 28 or transmit data to the web server 28, and the web server 28 may respond to such requests by obtaining the requested data and returning it to the user device or acting upon the transmitted data (e.g., storing posted data or executing posted commands). In some embodiments, the requests are for a new webpage or for data upon which client-side scripts will base changes in the webpage, such as XMLHttpRequest requests for data in a serialized format, e.g. JavaScript™ object notation (JSON) or extensible markup language (XML). The web server 28 may communicate with web browsers, such as the web browser 42 or 44 executed by user devices 36 or 38. In some embodiments, the webpage is modified by the web server 28 based on the type of user device, e.g., with a mobile webpage having fewer and smaller images and a narrower width being presented to the mobile user device 36, and a larger, more content rich webpage being presented to the desk-top user device 38. An identifier of the type of user device, either mobile or non-mobile, for example, may be encoded in the request for the webpage by the web browser (e.g., as a user agent type in an HTTP header associated with a GET request), and the web server 28 may select the appropriate interface based on this embedded identifier, thereby providing an interface appropriately configured for the specific user device in use.

The illustrated data store 30, in some embodiments, stores patient medical information (e.g., in the form of an electronic medical record); patient biographical information; clinical measurements; test results; sensor data; cardiovascular waveforms, indices, and/or data indicative of such waveforms and/or indices; reduced-order parameters associated with one or more of the waveforms weights associated with different types of information; relational data; artificial intelligence models and/or instructions for training and/or executing such models; and/or other information. Data store 30 may include various types of data stores, including relational or non-relational databases, document collections, hierarchical key-value pairs, or memory images, for example. Such components may be formed in a single database, document, or the like, or may be stored in separate data structures. In some embodiments, data store 30 comprises electronic storage media that electronically stores information. The electronic storage media of data store 30 may include one or both of system storage that is provided integrally (i.e., substantially non-removable) with the system 10 and/or removable storage that is removably connectable to the system 10 via, for example, a port (e.g., a USB port, a firewire port, etc.) or a drive (e.g., a disk drive, etc.). Data store 30 may be (in whole or in part) a separate component within the system 10, or data store 30 may be provided (in whole or in part) integrally with one or more other components of the system 10 (e.g., controller 14, etc.). In some embodiments, data store 30 may be located in a data center, in a server that is part of external resources 46, in a computing device 34, 36, or 38, and/or in other locations. Data store 30 may include one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), or other electronically readable storage media. Data store 30 may store software algorithms, information determined by controller 14, information received via the graphical user interface displayed on computing devices 34, 36, and/or 38, information received from external resources 46, or other information accessed by system 10 to function as described herein. For example, in some embodiments, some or all of the above described information may be automatically obtained by transfer engine 12 from one or more electronically accessible databases. These databases may be provided within and/or outside of system 10 (e.g., by data store 30 and/or external resources 46). The information may be automatically obtained based on instructions provided by a user through a user interface (as described herein).

Controller 14 is configured to coordinate the operation of the other components of transfer engine 12 to provide the functionality described herein. Controller 14 may be formed by one or more processors, for example. In some embodiments, controller 14 may be configured to control different aspects of the functionality described herein based on different individual programming components (though these components are not specifically illustrated in FIG. 1). Controller 14 may be configured to direct the operation of components by software; hardware; firmware; some combination of software, hardware, or firmware; or other mechanisms for configuring processing capabilities. In some embodiments, transfer engine 12 (e.g., controller 14 in addition to cache server 32, web server 28, and/or API server 26) is executed in a single computing device, or in a plurality of computing devices in a datacenter, e.g., in a service oriented or micro-services architecture.

Controller 14 is configured to receive input(s) for a trained sequentially-reduced AI model. The input(s) may comprise patient data having one, two, or more cardiovascular waveforms (i.e., radial and/or brachial pressure or vessel wall displacement waveforms in any order). The waveforms may be broken down to 50-5000 or more discrete datapoints, for example. In some embodiments, the one, two, or more waveforms are from a pulse oximeter measurement or comprise a femoral waveform, for example.

In some embodiments, the AI model and/or AI model architecture comprises one or more artificial neural networks (ANNs), feedforward neural networks (FNNs), recurrent neural networks (RNNs), temporal convolutional neural networks (TCNNs), Random Forest Regressors (RFRs), and/or other architectures. In some embodiments, the trained AI model comprises an ANN. In some embodiments, the trained AI model comprises an FNN. In some embodiments, the trained AI model comprises an RNN, a TCNN, or an RFR, for example. In some embodiments, the trained AI model comprises any architecture configured to efficiently execute the operations described herein. In some embodiments, the AI model may be a machine learning algorithm. In some embodiments, the machine learning algorithm may be or include a neural network (e.g., one of the example neural networks listed above), classification tree, decision tree, support vector machine, or other model that is trained and configured to function as described herein.

As an example, neural networks may be based on a large collection of neural units (or artificial neurons). Neural networks may loosely mimic the manner in which a biological brain works (e.g., via large clusters of biological neurons connected by axons). Each neural unit of a neural network may be simulated as being connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

In some embodiments, controller 14 is configured to determine, via the trained sequentially-reduced AI model, from one or more of the one or more cardiovascular waveforms, a pressure waveform corresponding to the carotid artery (or vessel wall displacement waveform of the carotid artery). In some embodiments, the determining comprises determining, utilizing the trained AI model, from one, two, or more waveforms, the patient's cardiovascular indices such as cardiac output, carotid-femoral pulse wave velocity, LV stroke volume, LV filling pressure (e.g., LV end diastolic pressure), LV contractility (e.g., LV ejection fraction, fractional shortening, LV end systolic elastance), aortic characteristic impedance, arterial compliance, LV compliance, and LV-aortic coupling indices as well as cardiovascular-affecting disease indices such as HOMA index (for diabetes). Controller 14 is configured to, responsive to determining the carotid waveform and/or the cardiovascular indices, provide, to a user (e.g., a medical practitioner such as a doctor and/or other users), underlying pathology information revealed by carotid artery information indicated by the pressure waveform corresponding to the carotid artery and/or the cardiovascular indices.

In some embodiments, patient data having one, two, or more cardiovascular waveforms comprises: radial pressure waveforms, brachial pressure waveforms, carotid pressure waveforms, radial vessel wall displacement waveforms, brachial vessel displacement waveforms, carotid vessel displacement waveforms, and/or other waveforms. In some embodiments, the one or two input waveforms to the sequentially-reduced AI model are inputted in different orders. The different orders may comprise: (i) only radial, (ii) only brachial, (iii) first radial, then brachial, and (iv) first brachial, then radial, for example (the input order does not matter here).

In some embodiments, the one, two, or more cardiovascular waveforms comprise patient data having two or more cardiovascular waveforms, such as radial and/or brachial and/or carotid pressure and/or vessel wall displacement waveforms in any order. In some embodiments, the one, two, or more input waveforms to the sequentially-reduced AI model are inputted in different orders. The different orders may comprise at least one of: (i) first radial, second brachial, (ii) first brachial, second radial, (iii) first radial, second carotid, (iv) first carotid, second radial, (v) first brachial, second carotid, (vi) first carotid, second brachial, (vii) first radial, second brachial, third carotid, (viii) first radial, second carotid, third brachial, (ix) first brachial, second radial, third carotid, (x) first brachial, second carotid, third radial, (xi) first carotid, second radial, third brachial, or (xii) first carotid, second brachial, third radial.

In some embodiments, the outputs of the sequentially-reduced AI model are the reduced-order parameters corresponding to at least one of: the carotid pressure waveform or vessel wall displacement of the carotid artery, the reduced-order parameters including intrinsic frequencies, augmentation indices, wave intensity parameters, form factor, pulse pressure amplification, travel time of the reflected wave, or Fourier transform representation components.

In some embodiments, the intrinsic frequencies comprise the double frequency version or the multiple harmonic intrinsic frequency version. Such advanced intrinsic frequencies divide the output of the heart-aorta-brain system (e.g., carotid pressure waveforms) into multiple distinct phases throughout the cardiac cycle, each governed by a different dynamical system. For example, in the triple version of the intrinsic frequency, the carotid waveform phases include the a) inotropic phase where the dynamics is dominated by the LV; b) volume displacement phase when the cerebral vasculature, aorta and LV are strongly coupled together; and c) decoupling phase when the LV is decoupled from the aorta and cerebral vasculature due to the closure of the aortic valve.

In some embodiments, the wave intensity parameters (of a given waveform) comprise at least one of a first forward peak/time, a first backward peak/time, or a second forward peak/time, for example. Wave intensity (WI) analysis is a well-established pulse wave analysis technique for quantifying the energy carried by arterial waves. WI is determined by incremental changes in pressure and flow velocity, and hence requires measurements of both. A typical pattern of WI consists of a large amplitude forward (positive) peak (corresponding to the initial compression caused by a left ventricular contraction) followed by a small amplitude backward (negative) peak (corresponding to reflections from the initial contraction) which itself may be followed by a moderate amplitude forward decompression wave.

In some embodiments, the inputs are the reduced-order representation of the waveforms using any basis function expansion, Fourier transform representation, or the intrinsic frequency representation of the waveform(s). In some embodiments, the basis function expansion comprises eigenfunctions. In some embodiments, the Fourier transform representation is truncated by one or more different frequencies. In some embodiments, any short-time Fourier transform, windowed Fourier transform, or wavelet transform is used to provide as input such reduced-order representations and expansions based on subdivided segments of a waveform.

In some embodiments, controller 14 is configured to train the (sequentially-reduced) AI model. Controller 14 is configured to cause the AI model to learn to transfer or convert one waveform to another, and/or to reduced-order parameters of another anatomical site's waveform, and/or perform other functions as described herein. In some embodiments, controller 14 is configured to train the model with a training algorithm (e.g., Levenberg-Marquard and/or other training algorithms) using a combination of synthetic data (e.g., numerically simulated), preclinical data, clinical data measured by different devices, and/or other data. A custom loss function based on the reduce-order parameters may be implemented (if needed) during the model training process.

Controller 14 may be configured to, during steps for model training, utilize Fourier-based custom loss functions designed to incorporate a reconstructed weighted waveform, a waveform's second derivative weighted or, the reconstructed waveform's second derivative weighted reduced-order parameters from input and output waveforms, or combinations thereof. In some embodiments, Fourier transform representation components comprise the amplitude and/or phase of sinusoidal components with different frequencies. In some embodiments, reduced-order parameters utilized in Fourier-based custom loss functions comprise any number of reduced-order parameters (e.g., any number components from the Fourier transform representation components from the first 10 to the first 25 components, or any other desired number). In some embodiments, input and output waveforms utilized in the Fourier-based custom loss functions for generating reduced-order parameters comprise patient data having two or more cardiovascular waveforms including radial and/or brachial and/or carotid pressure and/or vessel wall displacement waveforms, in any order, for example.

In some embodiments, controller 14 is configured to implement short-time Fourier transform-based or windowed Fourier-transform-based representation methods during generating of reduced-order parameters utilized in Fourier-based custom loss functions. In some embodiments, controller 14 is configured to apply short-time Fourier transform-based or windowed Fourier-transform-based methods on any segment of a waveform, including diastolic, systolic, or any other desired time-interval or subdivided segment within the cardiac cycle.

In some embodiments, controller is configured to provide the trained AI model to a client device (e.g., a mobile user device 34 and/or a desktop user device 38, other client device(s) 20, etc.) having a diagnosis module configured to execute the trained AI model to perform the determination of a specific cardiovascular disease via the client device. The client device may be a smartphone, microwave-based device, a wearable device, and/or other devices as described above. In some embodiments, the client device includes at least one sensor (e.g., other client device 20 shown in FIG. 1) configured to measure an arterial blood pressure of a patient, a pulse rate of the patient, a pulse-ox of the patient, an arterial wall displacement of the patient, and/or other parameters. In some embodiments, the client device comprises a sensor such as an electrocardiogram (ECG) device configured to capture an ECG of the patient. In some embodiments, the client device is an implantable wireless system. In some embodiments, the client device is an invasive arterial line and/or other devices.

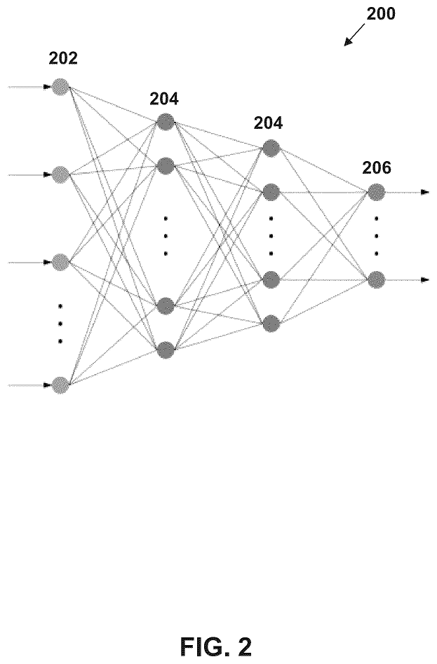

FIG. 2-8, and the corresponding discussion below, further elaborate on the functionality of controller 14 and/or other components of system 100 described above. For example, FIG. 2 illustrates an example of a sequentially reduced feedforward neural network 200 (an example of an AI model described above). As described above, neural network 200 may be or include one or more artificial neural networks (ANNs), recurrent neural networks (RNNs), temporal convolutional neural networks (TCNNs), Random Forest Regressors (RFRs), feedforward neural networks (FNNs), and/or other architectures. An example FNN is shown in FIG. 2. In some embodiments, the example FNN (neural network 200) comprises an input layer 202, several hidden layers 204, and an output layer 206.

Various other types of machine learning models may be implemented such as, for example, Ordinary Least Squares Regression (OLSR), Linear Regression, Logistic Regression, Stepwise Regression, Multivariate Adaptive Regression Splines (MARS), Locally Estimated Scatterplot Smoothing (LOESS), Instance-based Algorithms, k-Nearest Neighbor (KNN), Learning Vector Quantization (LVQ), Self-Organizing Map (SOM), Locally Weighted Learning (LWL), Regularization Algorithms, Ridge Regression, Least Absolute Shrinkage and Selection Operator (LASSO), Elastic Net, Least-Angle Regression (LARS), Decision Tree Algorithms, Classification and Regression Tree (CART), Iterative Dichotomizer 3 (ID3), C4.5 and C5.0 (different versions of a powerful approach), Chi-squared Automatic Interaction Detection (CHAID), Decision Stump, M5, Conditional Decision Trees, Naive Bayes, Gaussian Naive Bayes, Causality Networks (CN), Multinomial Naive Bayes, Averaged One-Dependence Estimators (AODE), Bayesian Belief Network (BBN), Bayesian Network (BN), k-Means, k-Medians, K-cluster, Expectation Maximization (EM), Hierarchical Clustering, Association Rule Learning Algorithms, A-priori algorithm, Eclat algorithm, Artificial Neural Network Algorithms, Perceptron, Back-Propagation, Hopfield Network, Radial Basis Function Network (RBFN), Deep Learning Algorithms, Deep Boltzmann Machine (DBM), Deep Belief Networks (DBN), Convolutional Neural Network (CNN), Deep Metric Learning, Stacked Auto-Encoders, Dimensionality Reduction Algorithms, Principal Component Analysis (PCA), Principal Component Regression (PCR), Partial Least Squares Regression (PLSR), Collaborative Filtering (CF), Latent Affinity Matching (LAM), Cerebri Value Computation (CVC), Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA), Ensemble Algorithms, Boosting, Bootstrapped Aggregation (Bagging), AdaBoost, Stacked Generalization (blending), Gradient Boosting Machines (GBM), Gradient Boosted Regression Trees (GBRT), Random Forest, Computational intelligence (evolutionary algorithms, etc.), Computer Vision (CV), Natural Language Processing (NLP), Recommender Systems, Reinforcement Learning, Graphical Models, separable convolutions (e.g., depth-separable convolutions, spatial separable convolutions, etc.), and/or other models.

In some embodiments, waveforms representing a cardiac cycle of a patient or patients may be obtained (e.g., by controller 14 shown in FIG. 1 and described above). For example, a blood pressure waveform may be obtained via a client device (e.g., a smartphone or other mobile user device 34 shown in FIG. 1; a sensor, wearable, or other client device 20 shown in FIG. 1; etc.). In some embodiments, one or more operations described herein may be executed on the client device, on a computing system, or some of the process may be executed on the client device and some of the process may be executed on the computing system (e.g., as also described elsewhere in this document).

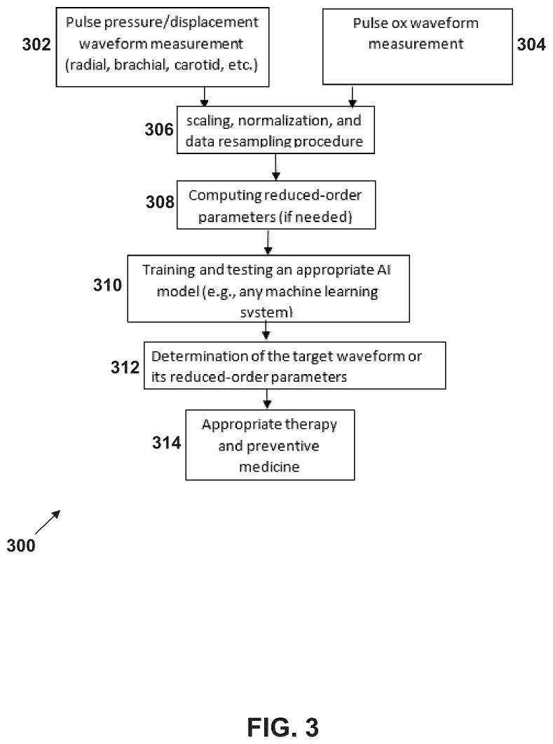

For example, FIG. 3 shows an example block diagram of operations 300 for applying cardiovascular transfer functions in accordance with various embodiments. In a first operation 302 and/or 304, waveforms representing a cardiac cycle of a patient or patients may be obtained by controller 14 shown in FIG. 1 and described above. For example, a blood pressure/displacement waveform may be obtained via a client device (e.g., a smartphone or other mobile user device 34 shown in FIG. 1), and/or a pulse ox waveform measurement may be obtained (e.g., via pulse ox sensor, or a wearable, or other client device 20 shown in FIG. 1), and provided to controller 14.

As several additional examples, systems, components, devices, sensors, and/or other hardware components, or other software based instructions (e.g., other client devices 20 shown in FIG. 1), can be used to measure the waveforms (e.g., a smartwatch configured to measure an arterial blood pressure of a patient and generate an arterial blood pressure waveform representing the measured arterial blood pressures of the patient). This may include a portable electronic hemodynamic sensor systems that can measure hemodynamic waveforms. This may include a smartphone application and system, with or without electrocardiogram (ECG) ability, that can be used to measure pulse waveforms for IF parameters (as described herein) and intrinsic phases, and pre-ejection period (PEP) for a cardiac triangle mapping (CTM) method. (Additional details regarding the CTM methodology, including clinical efficacy of the technique, is included within “Cardiac Triangle Mapping: A New Systems Approach for Noninvasive Evaluation of Left Ventricular End Diastolic Pressure,” Pahlevan et al., Fluids 2019, 4, 16, the contents of which are incorporated herein by reference in their entireties.) This may include optical sensors that can measure vessel wall motions. This may include tonometry devices that can measure pressure waveforms; microwave devices that can measure vessel wall motions; echo ultrasound devices that can measure vessel wall motions; implanted pressure sensors in the large systemic vessels; inline and invasive radial or femoral catheters; a computer system (e.g., system 10 shown in FIG. 1, or components thereof) that automatically perform required computations from acquired signals, an ECG system, and/or other devices (e.g., client devices 20 shown in FIG. 1).

In a second operation 306, scaling, normalization, and data resampling techniques can be applied (e.g., by controller 14 shown in FIG. 1) to the waveforms in order to reconcile the data over different species (different physiological states) and different measurement devices (different sampling rates and measurement units). In a third operation 308, reduced-order parameters (if needed) may be determined (by controller 14) from the prepared arterial (pressure or diameter) waveform, and/or other information.

In a fourth operation 310, an AI model (e.g., an FNN, an ANN, etc., as described above) including input layers (e.g., for waveform signal and/or other waveform parameters), hidden layers (e.g., with at least two neurons/nodes), and output layers (e.g., with at least one neuron/node revealing the target waveform) may be selected, trained, tested, and used. As described above, the AI model can be trained (by controller 14 shown in FIG. 1) with a training algorithm (e.g., Levenberg-Marquard and/or other training algorithms) using a combination of synthetic data (e.g., numerically simulated), preclinical data, clinical data measured by different devices, and/or other data. A custom loss function based on the reduce-order parameters may be implemented (if needed) during the model training process. The trained AI model may be (blindly) tested (e.g., AI model validation) on additional cases/subjects to ensure the accuracy of the model. In some embodiments, if the accuracy of the model after training is less than a threshold accuracy (e.g., 85% accurate or more, 95% accurate or more, etc.), then the model may be re-trained using the same, updated, or new training data.

In a fifth operation 312, a determination of a target waveform or its reduced-order parameters may be made (e.g., an input waveform may be transferred or converted into a second (target) waveform by controller 14 shown in FIG. 1). In a sixth operation 314, appropriate therapy and/or preventive medicine (as two examples) may be determined for a patient based on the target waveform (e.g., automatically by controller 14, manually by a doctor and/or other practitioners, etc.)

As described above, data (e.g., with which the parameters to be input to the trained machine learning model are derived) can be measured (e.g., at operations 302 and/or 304 shown in FIG. 3) using non-invasively and instantaneously (or nearly instantaneously) using portable devices (e.g., mobile user devices 34 and/or other client devices 20 shown in FIG. 1 and described above). For example, a smartphone, a wearable device (e.g., smartwatch, smart bracelet, smart ring, etc.) may be used to capture the data (e.g., blood pressure measurements, electrocardiogram (ECG) data, pulse rate data, left ventricular end-diastolic pressure (LVEDP) values, etc.).

As described in International Application Nos. PCT/IB2021/059733 and PCT/IB2021/059735, titled “Noninvasive Cardiovascular Event Detection” and “Noninvasive Infarct Size Determination,” respectively, each of which were filed on Oct. 21, 2021, the disclosures of both are hereby incorporated by reference in their entireties, carotid waveforms may be captured using non-invasive techniques and the resulting pressure waves may be analyzed in an AI and/or machine learning setting. In some embodiments, transferring the temporal carotid pressure to its frequency domain counterpart may define a regression problem based on the recently introduced Intrinsic Frequencies (IF) methodology, which is described in greater detailed within “Noninvasive iPhone Measurement of Left Ventricular Ejection Fraction Using Intrinsic Frequency Methodology,” Pahlevan et al., Critical Care Medicine, 2017; 45:1115-1120, the contents of which is hereby incorporated by reference in its entirety.

To construct (i.e., train, validate, and test) the AI (e.g., FNN, ANN, etc.) model (e.g., at operation 310 shown in FIG. 3), an assortment of various carotid waveform signal sources may be used, including clinical databases (measured by various devices including Tonometry, Vivio, and iPhone) and a physiologically generated synthetic database. The synthetic database can ensure the mathematical training of the IF method. The clinical databases can enrich the training algorithm and subsequently the AI model for subsequent clinical purposes (e.g., preparation for real-world physiological variations and noises, which are not considered by the first intrinsic mode function (IMF) assumption of the IF method). Additionally, a portion of the clinical database may be utilized for a blind-test process, which may be a completely blind-test, to assess the robustness/accuracy of the trained model more deeply.

In some embodiments, one or more pre-processing steps may be performed on an input waveform and/or other input data (e.g., as part of operation 306 in FIG. 3). In some embodiments, Fourier transform-based reduced-order methodologies and representations may be used for pre-processing of the custom loss function-based AI-model training. In some embodiments, a waveform normalization procedure may be performed.

The IF method works with the shape (morphology) of an arterial pressure waveform, for example. Therefore, any device capable of recording the arterial waveform (e.g. smartphone, arterial applanation tonometry, etc.) with any arbitrary measurement unit is compatible with the IF method. Accordingly, a broad range of values even in different orders of magnitude may be recorded for the same arterial waveform depending on the measurement unit. Although the waveform shape and the IF parameters are not dependent on the unit of the recorded signal, when it comes to collecting, archiving, or analyzing a substantial number of datapoints for the IF method (e.g., machine learning, deep learning, etc.), it is highly effective to reduce the size of the archive without loss of generality. As new devices and techniques are developed for non-invasive waveform measurements, which might lead to different measurement units or ranges of future signal records, the techniques and methodologies described herein may be applied.

As such, some embodiments include a new standard coordinate system for the arterial waveforms through which measurements of different devices (or even different species) can fall within the same range of signals and IF parameters. In addition to the new standard coordinate system, a normalized time may also be proposed along with the new standard coordinate setup. These systems and techniques may generate the same cardiac cycle period (T′=1) for all the arterial waveforms. Such a coordinate system can be achieved for all the arterial waveforms (e.g., coming from different sensor platforms), thereby saving enormous storage and time (especially in big-data studies).

In some embodiments, the data normalization process may include the following steps: (1) the minimum value Pₘᵢₙ=P(t) of the signal (given in any arbitrary measuring unit) may be subtracted from the measured P(t) at all times of the entire cardiac cycle (i.e., P(t)−Pₘᵢₙ, 0≤t≤T); (2) the resulting waveform may be divided by its range over the entire cardiac cycle (i.e., \hat{P}(t)=(P(t)−Pₘᵢₙ)/(Pₘₐₓ−Pₘᵢₙ), 0≤t≤T)); (3) normalize in time by scaling t with the length T of the entire cardiac cycle (i.e., \hat{P}(τ)=\hat{P}(τ(t)), i=t/T, 0≤τ≤1). In these steps, T represents the full length (in time) of the cardiac cycle, and τ represents the normalized time.

The data produced via the data normalization process may lead to a scaled waveform (\hat{P}(τ)). From here, the IF method may be applied to the scaled waveform, and new (non-dimensional) IF parameters may be extracted as a result. The IF parameters may include a first intrinsic frequency ω₁ of a systolic portion of the cardiac cycle of the patient, a second intrinsic frequency ω₁ of a diastolic portion of the cardiac cycle, a systolic intrinsic phase angle φ₁, a diastolic intrinsic phase angle φ₂, a systolic envelope Rₛ, a diastolic envelope R_{d}, an envelope ratio (Rₛ/R_{d}), a relative height of the dicrotic notch (RHDN), an amount of time between a beginning of the systolic portion of the cardiac cycle and the dicrotic notch, an amount of time between a beginning of the systolic portion and an end of the diastolic portion, a maximum rate of change of a rising portion of the systolic portion of the cardiac cycle, or other parameters.

In some embodiments, a waveform resampling procedure may be performed. A candidate AI model (e.g., ANN, FNN, etc.) may be configured to receive the scaled carotid waveform as an input (as well as the dicrotic notch time), and produce the normalized IF parameters as model outputs. The AI model may import the discrete datapoints of a scaled carotid waveform. Different measurement devices may have different sampling rates and, even for a given measurement device, the cardiac cycle period may be different for different individuals, as well as for the same individual. In some cases, the normalized carotid waveforms may have different datapoint (vector) size. To obtain a global AI model, a fixed number of datapoints (e.g., N=500) may be used as the waveform size for input into the AI model. In some cases, the input (waveform) vectors are down/over-sampled, which may generate inputs of uniform dimension for the network. The generated inputs may enable usage of any measurement device for capturing pressure waveform measurements. As an example, the waveform down/over-sampling process may be performed using a spline interpolation to space R⁵⁰⁰. R⁵⁰⁰ is a space with 500 dimensions.

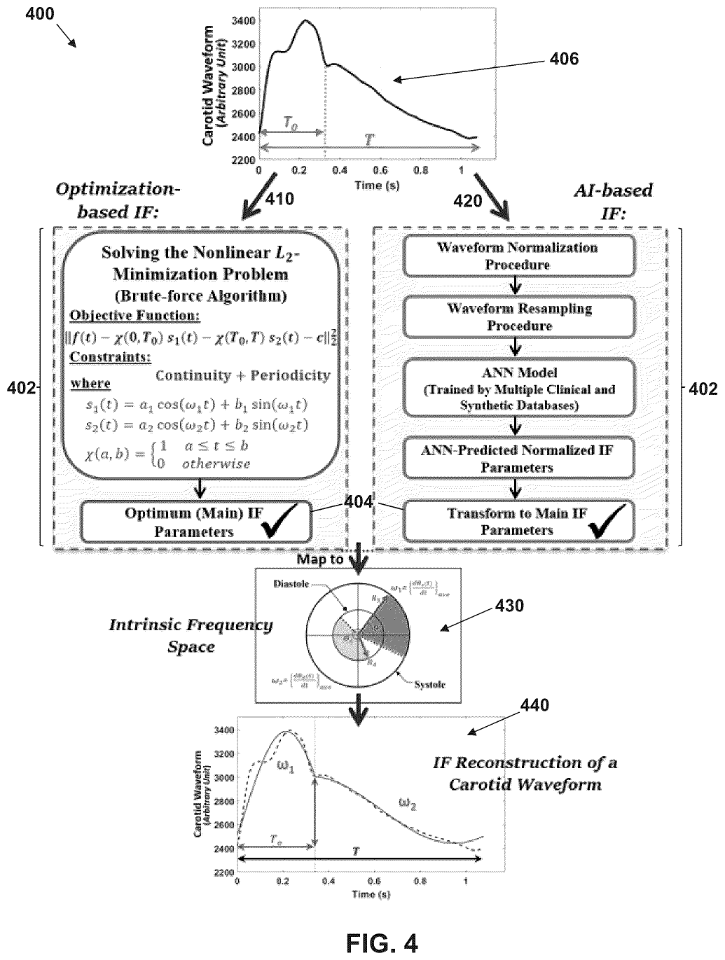

FIG. 4 illustrates a flowchart 400 describing determination 402 of IF methodology parameters 404 based on an input carotid waveform 406 (as one example input waveform). FIG. 4 compares an optimization based IF methodology parameter determination approach 410 with an AI based approach 420. An ANN model is used in this example. As shown in FIG. 4, once parameters 404 are determined, they are mapped to an intrinsic frequency space 430, and IF reconstruction 440 of a carotid waveform is performed. T₀ and T are the dicrotic notch time and the cardiac cycle period; c, a₁, a₂, b₁ and b₂ are the IF constants.

In some embodiments, training based on AI model results may be performed. In some cases, the input of the network may include the notch time and the interpolated waveform in space R⁵⁰¹, R⁵⁰¹ is R⁵⁰⁰⁺¹, and it is similar to R⁵⁰⁰ but the notch time is also added as an additional dimension to better extract the waveform's information. The output may include IF parameters, including ω₁, ω₂, R₁, θ₁, c. R₁ and c are the systolic envelope and the IF constant. The output variables may have different scales. Therefore, in cases where the output variables have different scales, it may be necessary to perform feature scaling to train a network that has close accuracy for all output variables. In some embodiments, the feature scaling for a variable y is defined as:

$$\hat{y} = \frac{y - mean ( y )}{std ( y )}$$

During training, weights and biases of the AI model (e.g., a neural network of the AI model) may be adjusted by minimizing a loss function. For example, a mean squared error (MSE) may be used as the loss function. In some embodiments, an L₁ or L₂ regularization may be added to the loss function to avoid over-fitting. An amount of regularization may be controlled using a hyper-parameter A. The optimal weights and biases may be obtained using the Adam stochastic optimizer, as one example. In each epoch, a training data set may be shuffled and then divided into several mini-batches. The weights and biases may be updated by minimizing the loss function on each mini-batch. In some cases, this updating may occur once. In some embodiments, the training is performed for a sufficient number of epochs to obtain a converged network. The convergence speed of the training can be controlled by the learning rate, (e.g., 10⁻³). In some embodiments, each network may be trained with one or more (e.g., up to 10 or more) restarts to avoid the influence of random initialization of weights and biases on the training.

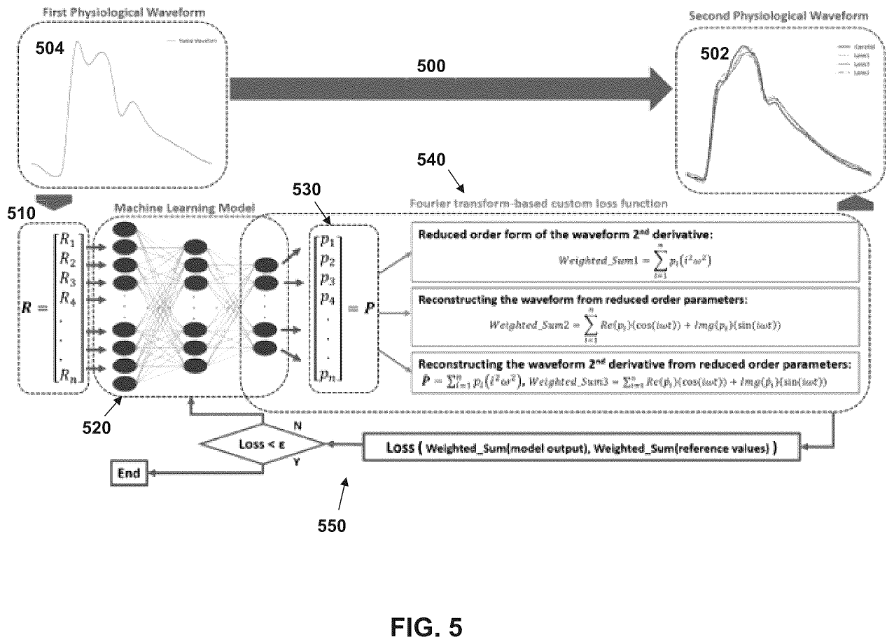

FIG. 5 illustrates an example of determining 500 a second (or target) waveform 502 based on a first (input) waveform 504. Determining 500 may be performed by controller 14 and/or other components of system 10 (shown in FIG. 1), as described herein. Determining 500 includes the operations described herein performed by controller 14 and/or other components of system 10, which may comprise or be thought of as a transfer function, for example. FIG. 5 illustrates determining a set 510 of parameters R (R₁, R₂, R₃, R₄, . . . , Rₙ)−Rᵢ here refers to the input reduced-order parameters e.g., discretized pressure waveform, the components of Fourier or windowed-Fourier transform representation of the waveform, which are input to an AI model 520 (in this example a machine learning model comprising a neural network). A set 530 of parameters P (p₁, p₂, p₃, p₄, . . . , pₙ)−Pᵢ here refers to the output reduced-order parameters e.g., discretized pressure waveform, the components of Fourier or windowed-Fourier transform representation of the waveform, output from model 520 is provided for Fourier transform-based custom loss function 540 loss determination 550, as shown in FIG. 5 and described above. Loss determination 500 may be repeated and model 520 re-trained and/or otherwise adjusted until the loss is less than a threshold amount c, for example. The loss functions in FIG. 5 are the physics-based weighted summations of the output (reduced-order parameters). For example, the loss functions can be derivative-based weighted (weight_sum1), reconstructed waveform-weighted, or reconstructed derivative-based waveform-weighted (weight_sum2 or weight_sum3).

In some embodiments, during the training process of an AI model, a Fourier transform-based custom loss function may be employed, as shown in FIG. 5. These custom loss functions are designed to incorporate weighted reduced-order parameters components derived from the reconstructed waveform, the waveform's second derivative, the reconstructed waveform's second derivative, or combinations thereof. FIG. 5 shows a flowchart diagram of an example of the training process using a Fourier transform-based custom loss function incorporating reduced-order parameters.

In some embodiments, a Fourier transform-based reduced-order method (e.g., Fourier transform-based representations) may be employed to generate reduced-order parameters. The input and output waveform (e.g., waveform 504 and waveform 502 in FIG. 5) for generating reduced-order parameters utilized in the Fourier-based custom loss functions may include radial and/or brachial and/or carotid pressure and/or any other desired cardiac waveform, for example. In some embodiments, the input to the AI model can comprise any number of reduced-order parameters (e.g., 1-n parameters are shown in set 510), such as components derived from the Fourier transform representation, ranging from the first 10 to the first 25 components, for example, or any other desired number.

In some embodiments, the implementation of short-time Fourier transform-based or windowed Fourier-transform-based representation methods is integrated into the process of generating reduced-order parameters used in the Fourier-based custom loss functions. Such methods can be applied to any time interval or subdivided segment within the complete cycle of a waveform, be it the diastolic interval, systolic interval, or any other desired time interval or segment within the cardiac cycle. Such methods can also be applied to the entire interval, on various subdivided segments, to provide frequency content that can be used as a reduced-order representation or compression of the original waveforms.

In some embodiments, wavelet transform-based methods may be used to perform the same decomposition and model reduction as above, but with multi-scale information.

In some embodiments, the training of AI model architectures, incorporating the implementation of Fourier-based custom loss functions, comprises a range of architectural possibilities. As described above, these architectures include, but are not limited to, artificial neural networks (ANN), feedforward neural networks (FNN), recurrent neural networks (RNN), temporal convolutional neural networks (TCNN), Random Forest Regressors (RFR), and other similar models and/or architectures. In some embodiments, different variations of Fourier transform-based custom loss functions may be implemented, incorporating features such as reconstructing waveform weighting, weighting by the waveform's second derivative, or weighting by the second derivative of the reconstructed waveform, based on reduced-order Fourier components from the inputs and outputs waveforms or combinations thereof.

Different quantities of hyper-parameters may be used for the training of the AI model. For example, the number hidden layers n_{L}∈[3,4,5]; the number of neurons of the first hidden layer n_{N}∈[512,256,128]; the regularization function f_{reg}∈[L₁, L₂]; and the regularization coefficient λ∈[10⁻⁵, 10⁻⁶, 10⁻⁷]. A grid-search of the hyper-parameters may be performed and/or other techniques may be used to find an optimal model configuration. The trained model that has the smallest validation error may be selected as the final model, for example. Additional analysis (e.g., PCA analysis) may be performed to ensure that the features of the model do not contribute too much to the model result. As one possible practical example of many potential embodiments, the AI model describe herein may comprise four hidden layers with 256, 128, 64, and 32 neurons, respectively, may be trained with the L₂ regularization, and the coefficient of the regularization may be 10⁻⁶. The training may be performed using the PyTorch machine learning library, for example.

Table 1 describes example generalization errors of a candidate model (where RMSE stands for root mean square error).

TABLE 1

| | | Relative | Max absolute |
|---|---|---|---|
| Output | RMSE | error(%) | error |
| ω1 | 0.769479 | 0.856751 | 5.326436 |
| ω2 | 1.331688 | 2.183796 | 12.688819 |
| R1 | 0.00619 | 0.85585 | 0.055652 |
| φ₁ | 0.013578 | 4.613535 | 0.1323 |
| C | 0.004977 | 1.499961 | 0.028939 |

As another example, the AI model described herein may comprise an ANN with six hidden layers with 32 neurons each, and may be trained with three examples of the above described Fourier-based custom loss function. The training may be performed using the PyTorch machine learning library and/or other information. Example generalization errors for this example ANN based AI model with Fourier-based custom loss function is presented in Table 2.

TABLE 2

| | | Pearson | |
|---|---|---|---|
| Output | R2Score | Correlation | MSE |
| Loss1 output Fourier components | 0.5637 | 0.9444 | 0.0031 |
| Loss2 output Fourier components | 0.3732 | 0.9154 | 0.0047 |
| Loss3 output Fourier components | 0.4846 | 0.9345 | 0.0037 |

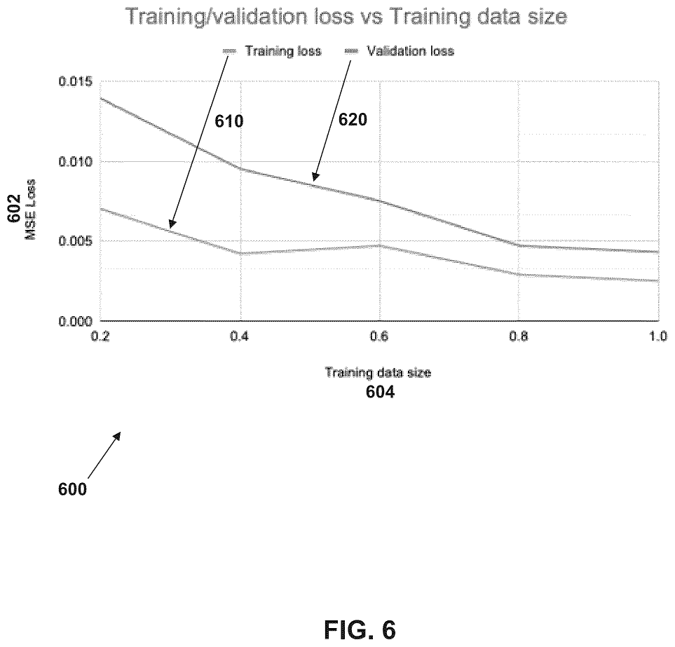

Training data set size may impact the accuracy of the AI model. FIG. 6 provides a graph 600 illustrating a sensitivity of the accuracy of an embodiment of the AI model described herein (in terms of mean square error (MSE) loss 602) to training data set size 604. FIG. 6 illustrates a training loss line 610, and a validation loss line 620, which both decrease with increasing training data set size 604.

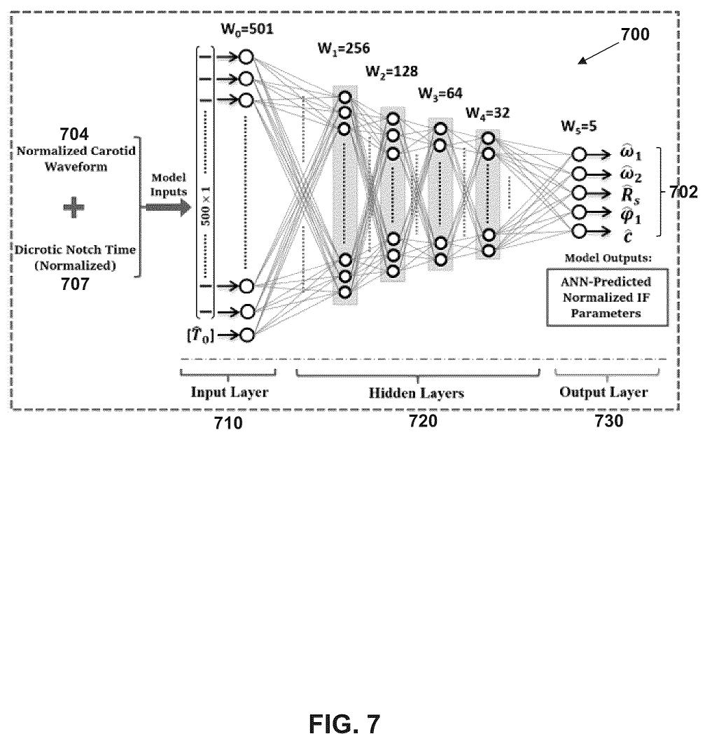

FIG. 7 is a schematic illustration of a trained sequentially-reduced AI (e.g., ANN in this example) model 700 for predicting (reduced-order) IF parameters 702 from a single input carotid pressure waveform 704 (e.g., 500 datapoints), and the dicrotic notch time 706, as two representative examples of an input waveform and reduced-order parameters, respectively. In some embodiments, \hat{T}₀ (which is normalized dicrotic notch time i.e., T₀/T) may also be input to model 700. In this example, AI model 700 comprises an input layer 710 with 501 neurons (W₀=501), four hidden layers 720 with 256, 128, 64, and 32 neurons, respectively (W₁=256, W₂=128, W₃=64, and W₄=32), and an output layer 730 with five neurons (W₅=5). Model 700 has a sequentially-reduced structure because the number of neurons in each layer systematically decreases from input layer 710 to output layer 730.

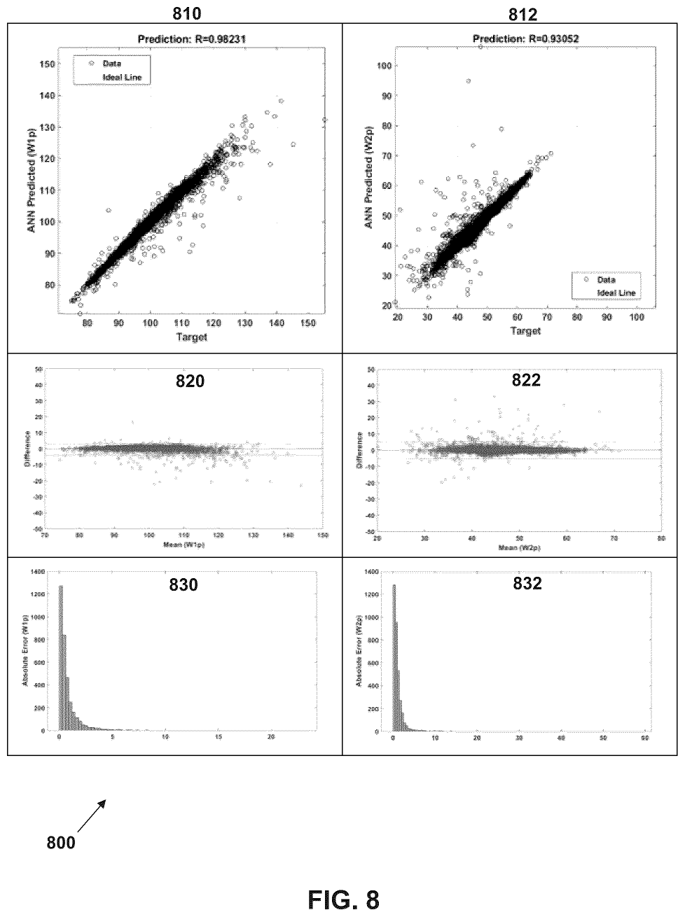

FIG. 8 illustrates example evaluation plots 800 for a representative embodiment of the AI model (e.g., model 700 shown in FIG. 7) described herein. Evaluation plots 800 include regression plots 810 and 812, Bland-Altman plots 820 and 822, and error histograms 830 and 832 for first and second scaled IFs (reduced-order parameters), respectively. This embodiment of the AI model was built and blindly tested on clinical data (e.g., 3472 clinical data points). Results from these plots are summarized in Table 3.

TABLE 3

| | | | Relative | Max absolute |
|---|---|---|---|---|
| Output | Range | RMSE | error(%) | error |
| ω1 | [74.966, 155.21] | 1.81 | 1.82 | 22.96 |
| ω2 | [19.247, 71.312] | 2.7 | 5.62 | 58.49 |
| R1 | [0.428, 0.888] | 0.01389 | 2.01 | 0.19 |
| φ₁ | [−1.269, −0.00825] | 0.03489 | 9.99 | 0.51 |
| C | [0.184, 0.5798] | 0.01444 | 4.00 | 0.19 |

The present application contemplates that the calculations disclosed in the embodiments herein may be performed in a number of ways, applying the same concepts taught herein, and that such calculations are equivalent to the embodiments disclosed.

Returning to FIG. 1, it should be noted that in some embodiments, transfer engine 12 may be configured such that in the above mentioned operations of the controller 14, input from users and/or sources of information inside or outside system 10 may be processed by controller 14 through a variety of formats, including clicks, touches, uploads, downloads, etc. The illustrated components (e.g., controller 14, API server 26, web server 28, data store 30, and cache server 32) of transfer engine 12 are depicted as discrete functional blocks, but embodiments are not limited to systems in which the functionality described herein is organized as illustrated by FIG. 1. The functionality provided by each of the components of transfer engine 12 may be provided by software or hardware modules that are differently organized than is presently depicted, for example such software or hardware may be intermingled, broken up, distributed (e.g. within a data center or geographically), or otherwise differently organized. The functionality described herein may be provided by one or more processors of one or more computers executing code stored on a tangible, non-transitory, machine readable medium.

FIG. 9 is a diagram that illustrates an exemplary computer system 900 in accordance with embodiments of the present system. Various portions of systems and methods described herein may include or be executed on one or more computer systems the same as or similar to computer system 900. For example, transfer engine 12, mobile user device 34, mobile user device 36, desktop user device 38, external resources 46 and/or other components of the system 10 (FIG. 1) may be and/or include one more computer systems the same as or similar to computer system 900. Further, processes, modules, processor components, and/or other components of system 10 described herein may be executed by one or more processing systems similar to and/or the same as that of computer system 900.

Computer system 900 may include one or more processors (e.g., processors 910a-910n) coupled to system memory 920, an input/output I/O device interface 930, and a network interface 940 via an input/output (I/O) interface 950. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computer system 900. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 920). Computer system 900 may be a uni-processor system including one processor (e.g., processor 910a), or a multi-processor system including any number of suitable processors (e.g., 910a-910n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computer system 900 may include a plurality of computing devices (e.g., distributed computer systems) to implement various processing functions.

I/O device interface 930 may provide an interface for connection of one or more I/O devices 960 to computer system 900. I/O devices may include devices that receive input (e.g., from a user) or output information (e.g., to a user). I/O devices 960 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 960 may be connected to computer system 900 through a wired or wireless connection. I/O devices 960 may be connected to computer system 900 from a remote location. I/O devices 960 located on a remote computer system, for example, may be connected to computer system 900 via a network and network interface 940.

Network interface 940 may include a network adapter that provides for connection of computer system 900 to a network. Network interface 940 may facilitate data exchange between computer system 900 and other devices connected to the network. Network interface 940 may support wired or wireless communication. The network may include an electronic communication network, such as the Internet, a local area network (LAN), a wide area network (WAN), a cellular communications network, or the like.

System memory 920 may be configured to store program instructions 970 or data 980. Program instructions 970 may be executable by a processor (e.g., one or more of processors 910a-910n) to implement one or more embodiments of the present techniques. Instructions 970 may include modules and/or components (e.g., components of controller 14 shown in FIG. 1) of computer program instructions for implementing one or more techniques described herein with regard to various processing modules and/or components. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 920 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 920 may include a non-transitory computer readable storage medium that may have program instructions stored thereon that are executable by a computer processor (e.g., one or more of processors 910a-910n) to cause the subject matter and the functional operations described herein. A memory (e.g., system memory 920) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times, e.g., a copy may be created by writing program code to a first-in-first-out buffer in a network interface, where some of the instructions are pushed out of the buffer before other portions of the instructions are written to the buffer, with all of the instructions residing in memory on the buffer, just not all at the same time.

I/O interface 950 may be configured to coordinate I/O traffic between processors 910a-910n, system memory 920, network interface 940, I/O devices 960, and/or other peripheral devices. I/O interface 950 may perform protocol, timing, or other data transformations to convert data signals from one component (e.g., system memory 920) into a format suitable for use by another component (e.g., processors 910a-910n). I/O interface 950 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 900 or multiple computer systems 900 configured to host different portions or instances of embodiments. Multiple computer systems 900 may provide for parallel or sequential processing/execution of one or more portions of the techniques described herein.

Those skilled in the art will appreciate that computer system 900 is merely illustrative and is not intended to limit the scope of the techniques described herein. Computer system 900 may include any combination of devices or software that may perform or otherwise provide for the performance of the techniques described herein. For example, computer system 900 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, a television or device connected to a television (e.g., Apple TV™), a Global Positioning System (GPS), a smartwatch, a wearable device, or the like. Computer system 900 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 900 may be transmitted to computer system 900 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present invention may be practiced with other computer system configurations.

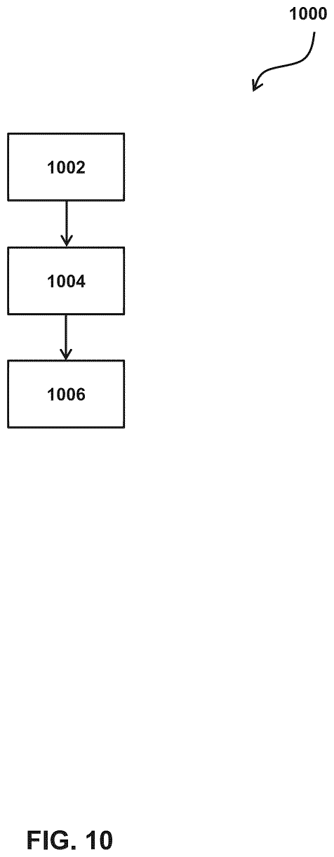

FIG. 10 is a flowchart that illustrates a sequentially-reduced artificial intelligence based process or method 1000 performed by the transfer engine and other components shown in FIG. 1 for cardiovascular transfer functions.

Method 1000 begins with receiving 1002, by inputs of a trained sequentially-reduced AI model (e.g., a feedforward neural network (FNN) model), patient data having one, two, or more cardiovascular waveforms (i.e., radial and/or brachial pressure or vessel wall displacement waveforms in any order) broken down to 50-5000 discrete datapoints, and/or its reduced-order parameters. In some embodiments, the one, two, or more waveforms are from a pulse oximeter measurement or comprise a femoral waveform, for example.

Method 1000 comprises determining 1004, via the trained sequentially-reduced AI model, from one, two, or more of the one or more cardiovascular waveforms, a pressure waveform corresponding to the carotid artery (or vessel wall displacement waveform of the carotid artery). In some embodiments, the determining 1004 comprises determining, utilizing the trained AI model, from two or more waveforms, the patient's cardiovascular indices such as cardiac output, carotid-femoral pulse wave velocity, LV stroke volume, LV filling pressure (e.g., LV end diastolic pressure), LV contractility (e.g., LV ejection fraction, fractional shortening, LV end systolic elastance), aortic characteristic impedance, arterial compliance, LV compliance, and LV-aortic coupling indices as well as cardiovascular-affecting disease indices such as HOMA index (for diabetes). Method 1000 comprises, responsive to determining the carotid waveform and/or the cardiovascular indices, providing 1006, to a user, underlying pathology information revealed by carotid artery information indicated by the pressure waveform corresponding to the carotid artery and/or the cardiovascular indices.

In some embodiments, patient data having one, two, or more cardiovascular waveforms comprises: radial pressure waveforms, brachial pressure waveforms, carotid pressure waveforms, radial vessel wall displacement waveforms, brachial vessel displacement waveforms, carotid vessel displacement waveforms, and/or other waveforms. In some embodiments, the one or two input waveforms to the sequentially-reduced AI model are inputted by or in different orders. The different orders may comprise: (i) only radial, (ii) only brachial, (iii) first radial, then brachial, and (iv) first brachial, then radial.

In some embodiments, the one, two, or more cardiovascular waveforms comprise patient data having two or more cardiovascular waveforms, such as radial and/or brachial and/or carotid pressure and/or vessel wall displacement waveforms in any order in any order. In some embodiments, the two or more input waveforms to the sequentially-reduced AI model are inputted in different orders. The different orders comprising at least one of: (i) first radial, second brachial, (ii) first brachial, second radial, (iii) first radial, second carotid, (iv) first carotid, second radial, (v) first brachial, second carotid, (vi) first carotid, second brachial, (vii) first radial, second brachial, third carotid, (viii) first radial, second carotid, third brachial, (ix) first brachial, second radial, third carotid, (x) first brachial, second carotid, third radial, (xi) first carotid, second radial, third brachial, or (xii) first carotid, second brachial, third radial.

In some embodiments, the outputs of the sequentially-reduced AI model are the reduced-order parameters corresponding to at least one of: the carotid pressure waveform or vessel wall displacement of the carotid artery. The reduced-order parameters may include intrinsic frequencies, augmentation indices, wave intensity parameters, form factor, pulse pressure amplification, travel time of the reflected wave, Fourier transform representation components, and/or other parameters.

In some embodiments, the intrinsic frequencies comprise the double frequency version or the multiple harmonic intrinsic frequency version.

In some embodiments, the wave intensity parameters comprise at least one of a first forward peak/time, a first backward peak/time, or a second forward peak/time.

In some embodiments, the inputs are the reduced-order representation of the waveforms using any basis function expansion, Fourier transform representation, or the intrinsic frequency representation of the waveform(s). In some embodiments, the basis function expansion comprises eigenfunctions. In some embodiments, the Fourier transform representation is truncated by one or more different frequencies. In some embodiments, any short-time Fourier transform, windowed Fourier transform, or wavelet transform is used to provide as input such reduced-order representations and expansions based on subdivided segments of a waveform.

In some embodiments, method 1000 comprises steps for training the trained sequentially-reduced AI model. In some embodiments, method 1000 comprises, during steps for model training, utilization of Fourier-based custom loss functions designed to incorporate a reconstructed weighted waveform, a waveform's second derivative weighted or, the reconstructed waveform's second derivative weighted reduced-order parameters from input and output waveforms, or combinations thereof.

In some embodiments, Fourier transform representation components comprise the amplitude and/or phase of the Sinusoidal components with different frequencies.

In some embodiments, model architecture comprises one or more FNNs, artificial neural networks (ANNs), recurrent neural networks (RNNs), temporal convolutional neural networks (TCNNs), Random Forest Regressors (RFRs), and/or other architectures. In some embodiments, the trained AI model comprises: a recurrent neural network (RNN), a temporal convolutional neural network (TCNN), or a Random Forest Regressor (RFR).

In some embodiments, reduced-order parameters utilized in Fourier-based custom loss functions comprise any number of reduced-order parameters (e.g., any number components from the Fourier transform representation components from the first 10 to the first 25 components, or any other desired number).

In some embodiments, input and output waveforms utilized in the Fourier-based custom loss functions for generating reduced-order parameters comprise patient data having two or more cardiovascular waveforms including: radial and/or brachial and/or carotid pressure and/or vessel wall displacement waveforms, in any order.

In some embodiments, method 1000 comprises implementation of short-time Fourier transform-based or windowed Fourier-transform-based representation methods during generating of reduced-order parameters utilized in Fourier-based custom loss functions. In some embodiments, method 1000 comprises application of short-time Fourier transform-based or windowed Fourier-transform-based methods on any segment of a waveform, including diastolic, systolic, or any other desired time-interval or subdivided segment within the cardiac cycle.

In some embodiments, method 1000 comprises providing the trained AI model to a client device having a diagnosis module configured to execute the trained AI model to perform the determination of a specific cardiovascular disease via the client device. The client device may be a smartphone, microwave-based device, or a wearable device. In some embodiments, the client device includes at least one sensor configured to measure an arterial blood pressure of a patient, a pulse rate of the patient, a pulse-ox of the patient, an arterial wall displacement of the patient. In some embodiments, the client device comprises an electrocardiogram (ECG) device configured to capture an ECG of the patient. In some embodiments, the client device is an implantable wireless system. In some embodiments, the client device is an invasive arterial line.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several inventions. Rather than separating those inventions into multiple isolated patent applications, applicants have grouped these inventions into a single document because their related subject matter lends itself to economies in the application process. But the distinct advantages and aspects of such inventions should not be conflated. In some cases, embodiments address all of the deficiencies noted herein, but it should be understood that the inventions are independently useful, and some embodiments address only a subset of such problems or offer other, unmentioned benefits that will be apparent to those of skill in the art reviewing the present disclosure. Due to cost constraints, some inventions disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such inventions or all aspects of such inventions.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device.

Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent application and eventual patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

The present techniques will be better understood with reference to the following enumerated embodiments, which may be combined in any combination.
1. A system comprising: at least one programmable processor; and a non-transitory machine-readable medium storing instructions which, when executed by the at least one programmable processor, cause at least one programmable processor to perform operations comprising: receiving, by inputs of a trained sequentially-reduced feedforward neural network (FNN) model, patient data having one or more cardiovascular waveforms, including radial and/or brachial pressure or vessel wall displacement waveforms in any order, broken down to 50-5000 discrete datapoints; determining, via the trained sequentially-reduced FNN model, from one or more of the one or more cardiovascular waveforms, a pressure waveform corresponding to a carotid artery, or vessel wall displacement waveform of the carotid artery; and responsive to determining the carotid waveform, providing, to a user, underlying pathology information revealed by carotid artery information indicated by the pressure waveform corresponding to the carotid artery.
2. The system of claim 1, wherein one or two input waveforms to the sequentially-reduced FNN model are inputted by different orders: (i) only radial; (ii) only brachial; (iii) first radial, then brachial; (iv) first brachial, then radial.
3. The system of embodiment 1, wherein outputs of the sequentially-reduced FNN model are reduced-order parameters corresponding to the carotid pressure waveform, or vessel wall displacement of the carotid artery, including intrinsic frequencies, either of a double frequency version or multiple harmonic intrinsic frequency version, augmentation indices, wave intensity parameters, including a first forward peak/time, first backward peak/time, and second forward peak/time, form factor, pulse pressure amplification, and/or travel time of a reflected wave.
4. The system of any of the previous embodiments, wherein inputs comprise a reduced-order representation of the waveforms using any basis function expansion, including eigenfunctions, Fourier transform representation, truncated by any number of frequencies, or an intrinsic frequency representation of a waveform.
5. The system of any of the previous embodiments, the operations further comprising utilization of Fourier-based custom loss functions, the loss functions configured to incorporate weighted reduced-order parameter components from a reconstructed waveform, a waveform's second derivative, the reconstructed waveform's second derivative, or combinations thereof, based on input and output waveforms during training steps of the FNN model.
6. A system comprising: at least one programmable processor; and a non-transitory machine-readable medium storing instructions which, when executed by the at least one programmable processor, cause at least one programmable processor to perform operations comprising: receiving, as inputs of a trained sequentially-reduced artificial intelligence (AI) model, patient data having one or more cardiovascular waveforms including radial and/or brachial and/or carotid pressure and/or vessel wall displacement waveforms in any order, broken down to 50-5000 discrete datapoints; determining, utilizing the trained AI model, from one or more waveforms, a patient's cardiovascular indices such as cardiac output, carotid-femoral pulse wave velocity, LV stroke volume, LV filling pressure, LV end diastolic pressure, LV contractility, LV ejection fraction, fractional shortening, LV end systolic elastance, aortic characteristic impedance, arterial compliance, LV compliance, and LV-aortic coupling indices as well as cardiovascular-affecting disease indices such as HOMA index (for diabetes); and as a result determining underlying pathology information revealed by such parameters to a user.
7. The system of any of the previous embodiments, wherein one or more input waveforms to the sequentially-reduced AI model are inputted by different orders: first radial, second brachial; first brachial, second radial; first radial, second carotid; first carotid, second radial; first brachial, second carotid; first carotid, second brachial; first radial, second brachial, third carotid; first radial, second carotid, third brachial; first brachial, second radial, third carotid; first brachial, second carotid, third radial; first carotid, second radial, third brachial; or first carotid, second brachial, third radial.
8. The system of any of the previous embodiments, wherein the one or more waveforms are from a pulse oximeter measurement or include a femoral waveform.
9. The system of any of the previous embodiments, wherein the AI model comprises a feedforward neural network (FNN) model and/or another AI structure comprising a recurrent neural network (RNN), a temporal convolutional neural network (TCNN), or Random Forest Regressor (RFR) is used.
10. The system of any of the previous embodiments, wherein the system further comprises a client device having a diagnosis module that includes the trained AI model and determines a specific cardiovascular disease.
11. The system of any of the previous embodiments, wherein the client device is a smartphone, microwave-based device, or a wearable device.
12. The system of any of the previous embodiments, wherein the client device is an implantable wireless system.
13. The system of any of the previous embodiments, wherein the client device is an invasive arterial line.
14. The system of any of the previous embodiments, the operations further comprising utilization of Fourier-based custom loss functions, the loss functions configured to incorporate weighted reduced-order parameter components from a reconstructed waveform, a waveform's second derivative, the reconstructed waveform's second derivative, or combinations thereof, based on input and output waveforms during training steps of the AI model.
15. The system of any of the previous embodiments, the operations further comprising training of AI models with various architectures using the Fourier-based custom loss functions, the various architectures including artificial neural networks (ANNs), feedforward neural networks (FNNs), recurrent neural networks (RNNs), temporal convolutional neural networks (TCNNs), and/or Random Forest Regressors (RFRs).
16. The system of any of the previous embodiments, the operations further comprising utilization of reduced-order parameters in the Fourier-based custom loss functions, the reduced-order parameters encompassing any number of components, including those from a Fourier transform representation, ranging from a first 10 to a first 25 components.
17. The system of any of the previous embodiments, the operations further comprising using patient data with two or more cardiovascular waveforms as inputs and outputs in the Fourier-based custom loss functions, the two or more waveforms including radial and/or brachial and/or carotid pressure and/or vessel wall displacement waveforms, in any order.
18. The system of any of the previous embodiments, the operations further comprising incorporating implementation of short-time or windowed Fourier transform-based representation methods during generation of reduced-order parameters used in the Fourier-based custom loss functions.
19. The system of any of the previous embodiments, the operation further comprising application of short-time or windowed Fourier transform-based methods on any segment of a waveform, including diastolic, systolic, or any other desired time-interval or subdivided segment within a cardiac cycle.
20. The system of any of the previous embodiments, wherein any short-time Fourier transform, windowed Fourier transform, or wavelet transform is used to provide as input reduced-order representations and expansions based on subdivided segments of a waveform.
21. A non-transitory machine-readable medium storing instructions which, when executed by at least one programmable processor, cause at least one programmable processor to perform operations comprising: receiving, by inputs of a trained sequentially-reduced feedforward neural network (FNN) model, patient data having one or more cardiovascular waveforms, including radial and/or brachial pressure or vessel wall displacement waveforms in any order, broken down to 50-5000 discrete datapoints; determining, via the trained sequentially-reduced FNN model, from one or more of the one or more cardiovascular waveforms, a pressure waveform corresponding to a carotid artery, or vessel wall displacement waveform of the carotid artery; and responsive to determining the carotid waveform, providing, to a user, underlying pathology information revealed by carotid artery information indicated by the pressure waveform corresponding to the carotid artery.
22. The medium of embodiment 21, wherein one or two input waveforms to the sequentially-reduced FNN model are inputted by different orders: (i) only radial; (ii) only brachial; (iii) first radial, then brachial; (iv) first brachial, then radial.
23. The medium of any of the previous embodiments, wherein outputs of the sequentially-reduced FNN model are reduced-order parameters corresponding to the carotid pressure waveform, or vessel wall displacement of the carotid artery, including intrinsic frequencies, either of a double frequency version or multiple harmonic intrinsic frequency version, augmentation indices, wave intensity parameters, including a first forward peak/time, first backward peak/time, and second forward peak/time, form factor, pulse pressure amplification, and/or travel time of a reflected wave.
24. The medium of any of the previous embodiments, wherein inputs comprise a reduced-order representation of the waveforms using any basis function expansion, including eigenfunctions, Fourier transform representation, truncated by any number of frequencies, or an intrinsic frequency representation of a waveform.
25. The medium of any of the previous embodiments, the operations further comprising utilization of Fourier-based custom loss functions, the loss functions configured to incorporate weighted reduced-order parameter components from a reconstructed waveform, a waveform's second derivative, the reconstructed waveform's second derivative, or combinations thereof, based on input and output waveforms during training steps of the FNN model.
26. A non-transitory machine-readable medium storing instructions which, when executed by at least one programmable processor, cause at least one programmable processor to perform operations comprising: receiving, as inputs of a trained sequentially-reduced artificial intelligence (AI) model, patient data having one or more cardiovascular waveforms including radial and/or brachial and/or carotid pressure and/or vessel wall displacement waveforms in any order, broken down to 50-5000 discrete datapoints; determining, utilizing the trained AI model, from one or more waveforms, a patient's cardiovascular indices such as cardiac output, carotid-femoral pulse wave velocity, LV stroke volume, LV filling pressure, LV end diastolic pressure, LV contractility, LV ejection fraction, fractional shortening, LV end systolic elastance, aortic characteristic impedance, arterial compliance, LV compliance, and LV-aortic coupling indices as well as cardiovascular-affecting disease indices such as HOMA index (for diabetes); and as a result determining underlying pathology information revealed by such parameters to a user.
27. The medium of any of the previous embodiments, wherein one or more input waveforms to the sequentially-reduced AI model are inputted by different orders: first radial, second brachial; first brachial, second radial; first radial, second carotid; first carotid, second radial; first brachial, second carotid; first carotid, second brachial; first radial, second brachial, third carotid; first radial, second carotid, third brachial; first brachial, second radial, third carotid; first brachial, second carotid, third radial; first carotid, second radial, third brachial; or first carotid, second brachial, third radial.
28. The medium of any of the previous embodiments, wherein the one or more waveforms are from a pulse oximeter measurement or include a femoral waveform.
29. The medium of any of the previous embodiments, wherein the AI model comprises a feedforward neural network (FNN) model and/or another AI structure comprising a recurrent neural network (RNN), a temporal convolutional neural network (TCNN), or Random Forest Regressor (RFR) is used.
30. The medium of any of the previous embodiments, wherein the operations further comprise controlling a client device having a diagnosis module that includes the trained AI model and determines a specific cardiovascular disease.
31. The medium of any of the previous embodiments, wherein the client device is a smartphone, microwave-based device, or a wearable device.
32. The medium of any of the previous embodiments, wherein the client device is an implantable wireless system.
33. The medium of any of the previous embodiments, wherein the client device is an invasive arterial line.
34. The medium of any of the previous embodiments, the operations further comprising utilization of Fourier-based custom loss functions, the loss functions configured to incorporate weighted reduced-order parameter components from a reconstructed waveform, a waveform's second derivative, the reconstructed waveform's second derivative, or combinations thereof, based on input and output waveforms during training steps of the AI model.
35. The medium of any of the previous embodiments, the operations further comprising training of AI models with various architectures using the Fourier-based custom loss functions, the various architectures including artificial neural networks (ANNs), feedforward neural networks (FNNs), recurrent neural networks (RNNs), temporal convolutional neural networks (TCNNs), and/or Random Forest Regressors (RFRs).
36. The medium of any of the previous embodiments, the operations further comprising utilization of reduced-order parameters in the Fourier-based custom loss functions, the reduced-order parameters encompassing any number of components, including those from a Fourier transform representation, ranging from a first 10 to a first 25 components.
37. The medium of any of the previous embodiments, the operations further comprising using patient data with two or more cardiovascular waveforms as inputs and outputs in the Fourier-based custom loss functions, the two or more waveforms including radial and/or brachial and/or carotid pressure and/or vessel wall displacement waveforms, in any order.
38. The medium of any of the previous embodiments, the operations further comprising incorporating implementation of short-time or windowed Fourier transform-based representation methods during generation of reduced-order parameters used in the Fourier-based custom loss functions.
39. The medium of any of the previous embodiments, the operation further comprising application of short-time or windowed Fourier transform-based methods on any segment of a waveform, including diastolic, systolic, or any other desired time-interval or subdivided segment within a cardiac cycle.
40. The medium of any of the previous embodiments, wherein any short-time Fourier transform, windowed Fourier transform, or wavelet transform is used to provide as input reduced-order representations and expansions based on subdivided segments of a waveform.
41. A method comprising: receiving, by inputs of a trained sequentially-reduced feedforward neural network (FNN) model, patient data having one or more cardiovascular waveforms, including radial and/or brachial pressure or vessel wall displacement waveforms in any order, broken down to 50-5000 discrete datapoints; determining, via the trained sequentially-reduced FNN model, from one or more of the one or more cardiovascular waveforms, a pressure waveform corresponding to a carotid artery, or vessel wall displacement waveform of the carotid artery; and responsive to determining the carotid waveform, providing, to a user, underlying pathology information revealed by carotid artery information indicated by the pressure waveform corresponding to the carotid artery.
42. The method of embodiment 41, wherein one or two input waveforms to the sequentially-reduced FNN model are inputted by different orders: (i) only radial; (ii) only brachial; (iii) first radial, then brachial; (iv) first brachial, then radial.
43. The method of any of the previous embodiments, wherein outputs of the sequentially-reduced FNN model are reduced-order parameters corresponding to the carotid pressure waveform, or vessel wall displacement of the carotid artery, including intrinsic frequencies, either of a double frequency version or multiple harmonic intrinsic frequency version, augmentation indices, wave intensity parameters, including a first forward peak/time, first backward peak/time, and second forward peak/time, form factor, pulse pressure amplification, and/or travel time of a reflected wave.
44. The method of any of the previous embodiments, wherein inputs comprise a reduced-order representation of the waveforms using any basis function expansion, including eigenfunctions, Fourier transform representation, truncated by any number of frequencies, or an intrinsic frequency representation of a waveform.
45. The method of any of the previous embodiments, further comprising utilization of Fourier-based custom loss functions, the loss functions configured to incorporate weighted reduced-order parameter components from a reconstructed waveform, a waveform's second derivative, the reconstructed waveform's second derivative, or combinations thereof, based on input and output waveforms during training steps of the FNN model.
46. A method comprising: receiving, as inputs of a trained sequentially-reduced artificial intelligence (AI) model, patient data having one or more cardiovascular waveforms including radial and/or brachial and/or carotid pressure and/or vessel wall displacement waveforms in any order, broken down to 50-5000 discrete datapoints; determining, utilizing the trained AI model, from one or more waveforms, a patient's cardiovascular indices such as cardiac output, carotid-femoral pulse wave velocity, LV stroke volume, LV filling pressure, LV end diastolic pressure, LV contractility, LV ejection fraction, fractional shortening, LV end systolic elastance, aortic characteristic impedance, arterial compliance, LV compliance, and LV-aortic coupling indices as well as cardiovascular-affecting disease indices such as HOMA index (for diabetes); and as a result determining underlying pathology information revealed by such parameters to a user.
47. The method of any of the previous embodiments, wherein one or more input waveforms to the sequentially-reduced AI model are inputted by different orders: first radial, second brachial; first brachial, second radial; first radial, second carotid; first carotid, second radial; first brachial, second carotid; first carotid, second brachial; first radial, second brachial, third carotid; first radial, second carotid, third brachial; first brachial, second radial, third carotid; first brachial, second carotid, third radial; first carotid, second radial, third brachial; or first carotid, second brachial, third radial.
48. The method of any of the previous embodiments, wherein the one or more waveforms are from a pulse oximeter measurement or include a femoral waveform.
49. The method of any of the previous embodiments, wherein the AI model comprises a feedforward neural network (FNN) model and/or another AI structure comprising a recurrent neural network (RNN), a temporal convolutional neural network (TCNN), or Random Forest Regressor (RFR) is used.
50. The method of any of the previous embodiments, further comprising controlling a client device having a diagnosis module that includes the trained AI model and determines a specific cardiovascular disease.
51. The method of any of the previous embodiments, wherein the client device is a smartphone, microwave-based device, or a wearable device.
52. The method of any of the previous embodiments, wherein the client device is an implantable wireless system.
53. The method of any of the previous embodiments, wherein the client device is an invasive arterial line.
54. The method of any of the previous embodiments, further comprising utilization of Fourier-based custom loss functions, the loss functions configured to incorporate weighted reduced-order parameter components from a reconstructed waveform, a waveform's second derivative, the reconstructed waveform's second derivative, or combinations thereof, based on input and output waveforms during training steps of the AI model.
55. The method of any of the previous embodiments, further comprising training of AI models with various architectures using the Fourier-based custom loss functions, the various architectures including artificial neural networks (ANNs), feedforward neural networks (FNNs), recurrent neural networks (RNNs), temporal convolutional neural networks (TCNNs), and/or Random Forest Regressors (RFRs).
56. The method of any of the previous embodiments, further comprising utilization of reduced-order parameters in the Fourier-based custom loss functions, the reduced-order parameters encompassing any number of components, including those from a Fourier transform representation, ranging from a first 10 to a first 25 components.
57. The method of any of the previous embodiments, further comprising using patient data with two or more cardiovascular waveforms as inputs and outputs in the Fourier-based custom loss functions, the two or more waveforms including radial and/or brachial and/or carotid pressure and/or vessel wall displacement waveforms, in any order.
58. The method of any of the previous embodiments, further comprising incorporating implementation of short-time or windowed Fourier transform-based representation methods during generation of reduced-order parameters used in the Fourier-based custom loss functions.
59. The method of any of the previous embodiments, further comprising application of short-time or windowed Fourier transform-based methods on any segment of a waveform, including diastolic, systolic, or any other desired time-interval or subdivided segment within a cardiac cycle.
60. The method of any of the previous embodiments, wherein any short-time Fourier transform, windowed Fourier transform, or wavelet transform is used to provide as input reduced-order representations and expansions based on subdivided segments of a waveform.

1. A system comprising:
at least one programmable processor; and a non-transitory machine-readable medium storing instructions which, when executed by the at least one programmable processor, cause at least one programmable processor to perform operations comprising:
receiving, by inputs of a trained sequentially-reduced feedforward neural network (FNN) model, patient data having one or more cardiovascular waveforms, including radial and/or brachial pressure or vessel wall displacement waveforms in any order, broken down to 50-5000 discrete datapoints;
determining, via the trained sequentially-reduced FNN model, from one or more of the one or more cardiovascular waveforms, a pressure waveform corresponding to a carotid artery, or vessel wall displacement waveform of the carotid artery; and
responsive to determining the carotid waveform, providing, to a user, underlying pathology information revealed by carotid artery information indicated by the pressure waveform corresponding to the carotid artery.

2. The system of claim 1, wherein one or two input waveforms to the sequentially-reduced FNN model are inputted by different orders: (i) only radial; (ii) only brachial; (iii) first radial, then brachial; (iv) first brachial, then radial.

3. The system of claim 1, wherein outputs of the sequentially-reduced FNN model are reduced-order parameters corresponding to the carotid pressure waveform, or vessel wall displacement of the carotid artery, including intrinsic frequencies, either of a double frequency version or multiple harmonic intrinsic frequency version, augmentation indices, wave intensity parameters, including a first forward peak/time, first backward peak/time, and second forward peak/time, form factor, pulse pressure amplification, and/or travel time of a reflected wave.

4. The system of claim 1, wherein inputs comprise a reduced-order representation of the waveforms using any basis function expansion, including eigenfunctions, Fourier transform representation, truncated by any number of frequencies, or an intrinsic frequency representation of a waveform.

5. The system of claim 1, the operations further comprising utilization of Fourier-based custom loss functions, the loss functions configured to incorporate weighted reduced-order parameter components from a reconstructed waveform, a waveform's second derivative, the reconstructed waveform's second derivative, or combinations thereof, based on input and output waveforms during training steps of the FNN model.

6. A system comprising:
at least one programmable processor; and a non-transitory machine-readable medium storing instructions which, when executed by the at least one programmable processor, cause at least one programmable processor to perform operations comprising:
receiving, as inputs of a trained sequentially-reduced artificial intelligence (AI) model, patient data having one or more cardiovascular waveforms including radial and/or brachial and/or carotid pressure and/or vessel wall displacement waveforms in any order, broken down to 50-5000 discrete datapoints;
determining, utilizing the trained AI model, from one or more waveforms, a patient's cardiovascular indices such as cardiac output, carotid-femoral pulse wave velocity, LV stroke volume, LV filling pressure, LV end diastolic pressure, LV contractility, LV ejection fraction, fractional shortening, LV end systolic elastance, aortic characteristic impedance, arterial compliance, LV compliance, and LV-aortic coupling indices as well as cardiovascular-affecting disease indices such as HOMA index (for diabetes); and
as a result determining underlying pathology information revealed by such parameters to a user.

7. The system of claim 6, wherein one or more input waveforms to the sequentially-reduced AI model are inputted by different orders:
first radial, second brachial;
first brachial, second radial;
first radial, second carotid;
first carotid, second radial;
first brachial, second carotid;
first carotid, second brachial;
first radial, second brachial, third carotid;
first radial, second carotid, third brachial;
first brachial, second radial, third carotid;
first brachial, second carotid, third radial;
first carotid, second radial, third brachial; or
first carotid, second brachial, third radial.

8. The system of claim 6, wherein the one or more waveforms are from a pulse oximeter measurement or include a femoral waveform.

9. The system of claim 6, wherein the AI model comprises a feedforward neural network (FNN) model and/or another AI structure comprising a recurrent neural network (RNN), a temporal convolutional neural network (TCNN), or Random Forest Regressor (RFR) is used.

10. The system of claim 6, wherein the system further comprises a client device having a diagnosis module that includes the trained AI model and determines a specific cardiovascular disease.

11. The system of claim 10, wherein the client device is a smartphone, microwave-based device, or a wearable device.

12. The system of claim 10, wherein the client device is an implantable wireless system.

13. The system of claim 10, wherein the client device is an invasive arterial line.

14. The system of claim 6, the operations further comprising utilization of Fourier-based custom loss functions, the loss functions configured to incorporate weighted reduced-order parameter components from a reconstructed waveform, a waveform's second derivative, the reconstructed waveform's second derivative, or combinations thereof, based on input and output waveforms during training steps of the AI model.

15. The system of claim 14, the operations further comprising training of AI models with various architectures using the Fourier-based custom loss functions, the various architectures including artificial neural networks (ANNs), feedforward neural networks (FNNs), recurrent neural networks (RNNs), temporal convolutional neural networks (TCNNs), and/or Random Forest Regressors (RFRs).

16. The system of claim 14, the operations further comprising utilization of reduced-order parameters in the Fourier-based custom loss functions, the reduced-order parameters encompassing any number of components, including those from a Fourier transform representation, ranging from a first 10 to a first 25 components.

17. The system of claim 14, the operations further comprising using patient data with two or more cardiovascular waveforms as inputs and outputs in the Fourier-based custom loss functions, the two or more waveforms including radial and/or brachial and/or carotid pressure and/or vessel wall displacement waveforms, in any order.

18. The system of claim 14, the operations further comprising incorporating implementation of short-time or windowed Fourier transform-based representation methods during generation of reduced-order parameters used in the Fourier-based custom loss functions.

19. The system of claim 18, the operation further comprising application of short-time or windowed Fourier transform-based methods on any segment of a waveform, including diastolic, systolic, or any other desired time-interval or subdivided segment within a cardiac cycle.

20. The system of claim 18, wherein any short-time Fourier transform, windowed Fourier transform, or wavelet transform is used to provide as input reduced-order representations and expansions based on subdivided segments of a waveform.

21-60. (canceled)
